(19) United States
(12) Patent Application Publication
(10) Pub. No.: US 2024/0082292 A1
(43) Pub. Date: Mar. 14, 2024

(54) NEURODEGENERATIVE TREATMENT

(71) Applicants: THE QUEEN'S UNIVERSITY OF BELFAST, Belfast (GB); UNIVERSITY OF ULSTER, Londonderry (GB)

(72) Inventors: Brian Desmond GREEN, Belfast (GB); Paula MCCLEAN, Londonderry (GB)

(21) Appl. No.: 18/141,286

(22) Filed: Apr. 28, 2023

Related U.S. Application Data
(63) Continuation-in-part of application No. PCT/GB2021/052818, filed on Oct. 29, 2021.
Application No. PCT/GB2021/052818 is a continuation-in-part of application No. PCT/GB2021/052817, filed on Oct. 29, 2021.

(30) Foreign Application Priority Data
Oct. 30, 2020 (GB) 2017251.6
Oct. 30, 2020 (GB) 2017255.7

(51) Int. Cl. A61K 31/715; A61K 36/185; A61P 25/28

(52) U.S. Cl. CPC A61K 31/715; A61K 36/185; A61P 25/28

(57) ABSTRACT

The invention is directed to isolated polysaccharide compositions, as well as use of the polysaccharide compositions in methods for treating a neurodegenerative disease or a medical condition associated therein.

### PRIORITY INFORMATION

This application is a continuation in part of International Application No. PCT/GB2021/052818, filed on Oct. 29, 2021, which claims the benefit of

Provisional Application Serial No. GB2017255.7, filed Oct. 30, 2020, and is also a continuation in part of International Application No. PCT/GB2021/052817, filed on Oct. 29, 2021, which claims the benefit of Provisional Application Serial No. GB2017251.6, filed on Oct. 30, 2020, the entire contents of each of which are incorporated by reference herein.

### SEQUENCE LISTING

The instant application contains a Sequence Listing which has been submitted electronically in XML format and is hereby incorporated by reference in its entirety. Said XML file, created on Aug. 3, 2023, is named “Neurodegenerative Treatment ST26 106533.xml” and is 2,887 bytes in size.

### FIELD OF THE INVENTION

The invention relates to an isolated polysaccharide and a composition comprising the polysaccharide for use in treating a neurodegenerative disease or a medical condition, and a method of treatment using the isolated polysaccharide or the composition thereof.

### BACKGROUND

Alzheimer's Disease (AD) is the most common cause of dementia and contributes to 60-70% of cases. AD is an incurable, neurodegenerative disease and its worldwide prevalence is expected to double every twenty years reaching well over 100 million by 2050. It is estimated that there will be approximately 14.8 million prevalent cases of AD in the US, Japan, and five major EU markets by 2034. Within the UK, there are approximately 850,000 sufferers which contribute an economic cost to society in the region of £23 bn. This is greater than heart disease and cancer combined. Drug development for AD has proven to be very difficult. The high-profile failure of clinical trials in AD, which primarily focus on the amyloid cascade hypothesis, strengthens the case for developing drugs with an alternative mechanism of action. Nevertheless, five drugs are currently approved for the treatment of AD including cholinesterase inhibitors (donepezil, rivastigmine, galantamine) and an N-methyl-D-aspartate (NMDA) receptor AD antagonist (memantine). These drugs alleviate some of the symptoms of the disease, but are not curative, or disease modifying, and only work in certain individuals for a relatively short period of time. No new treatments have been approved for AD since 2003. It is clear, therefore, that there is a significant unmet need for alternative, more effective, treatments for AD. There is therefore a need for a novel treatment of neurodegenerative diseases, such as Alzheimer's disease.

### STATEMENTS OF THE INVENTION

Thus, according to a first aspect of the invention, there is provided
an isolated polysaccharide comprising “n” repeating units; or
a composition comprising an isolated polysaccharide comprising “n” repeating units,
wherein each of the “n” repeating units comprises a backbone of alpha-(1-5)-linked arabinofuranose residues, a first side chain of a single alpha-arabinofuranose residue (1-2)-linked to an arabinofuranose residue of the backbone, and a second side chain of a single alpha-arabinofuranose residue (1-3)-linked to the same arabinofuranose residue of the backbone,
for use in treating, preventing or ameliorating a neurodegenerative disease and/or symptoms thereof in a subject,
optionally wherein the composition is a pharmaceutical composition and a pharmaceutically acceptable carrier or such like, or an edible composition.

According to another aspect, there is provided a method of treating, preventing or ameliorating a neurodegenerative disease and/or symptoms thereof in a subject, the method comprising administering to the subject an isolated polysaccharide referred to herein, a composition referred to herein, or a plant of the Malvales order or a part thereof.

Advantageously, the polysaccharide referred to herein is non-toxic and does not induce anaphylaxis in animals or humans. More importantly, however, the polysaccharide is edible and capable of crossing the blood brain barrier after oral consumption. Therefore, it can be administered orally to treat neurodegenerative diseases.

The method may comprise administering a therapeutically effective amount of the isolated polysaccharide, the composition or the plant to treat a neurodegenerative disease.

According to another aspect of the invention there is provided a plant of the Malvales order for use in treating, preventing or ameliorating a neurode generative disease and/or symptoms thereof in a subject.

The polysaccharide of the invention may be isolated from a Malvales plant. The plant may be a Malvales plant or a part thereof. Similarly, the polysaccharide may be isolated from a plant of the Malvales order. The Malvales order comprises the Bixaceae family, the Cistaceae family, the Cytinaceae family, the Dipterocarpaceae family, the Muntingiaceae family, the Neuradaceae family, the Sarcloaenaceae family, the Sphaerosepalaceae family, the Thymelaeceae family and the Malvaceae family. Preferably the plant is a member of the Malvaceae family. The family Malvaceae comprises the subfamilies Bombacoideae, Brownlowioideae, Bytnnerioideae, Byttnerioideae, Dombeyoideae, Grewioideae, Helicteroideae, Malvoideae, Sterculioideae and Tiliodeae. Preferably the plant is a Malvoideae. The Malvoideae comprises the tribes Malveae, Gossypieae, Hibisceae, Kydieae. Preferably the plant is from the Malveae tribe. The plant may be from the Sida genus or the Malva genus or the Malvastrum genus or the Sidalcea genus or the Abufilon genus or the Althea genus or the Sphaeralcea genus or the Lavatera genus. Most preferably the plant of the Sida genus is Sida cordifolia (also referred to as ilima, flannel weed, bala, country mallow or heart-leaf sida). Most preferably the plant of the Malva genus is Malva sylvestris. Most preferably the plant of the Malvastrum genus is Malvastrum laterifium. Most preferably the plant of the Sidalcea genus is Sidalcea malviflora. Most preferably plant of the Abufilon genus is Abufilon theophrasti. Most preferably the plant of Althea genus is Althea officinalis. Most preferably the plant of Sphaeralcea genus is Sphaeralcea coccinea. Most preferably the plant of Lavatera genus is Lavatera arborea. Most preferably the plant is Sida cordifolia.

Thus, the polysaccharide may be from a plant of the Sida genus (e.g. Sida cordifolia) or the Malva genus (e.g. Malva sylvestris) or the Malvastrum genus (e.g. Malvastrum laterifium) or the Sidalcea genus (e.g. Sidalcea malviflora) or the Althea genus (e.g. Althea officinalis), or the Sphaeralcea genus (e.g. Sphaeralcea coccinea), or the Lavatera genus (e.g. Lavatera arborea). Similarly, the plant may be a plant of the Sida genus (e.g. Sida cordifolia) or the Malva genus (e.g. Malva sylvestris) or the Malvastrum genus (e.g. Malvastrum laterifium) or the Sidalcea genus (e.g. Sidalcea malviflora) or the Althea genus (e.g. Althea officinalis), or the Sphaeralcea genus (e.g. Sphaeralcea coccinea), or the Lavatera genus (e.g. Lavatera arborea).

Preferably the polysaccharide is from Sida cordifolia. Preferably the plant is Sida cordifolia. The polysaccharide may be isolated from part of a plant of the Malvales order, such as the leaves, the flower, the stem and/or the roots of the plant. The plant may be part of the plant, such as the leaves, the flower, the stem and/or the roots of the plant. Preferably the part of the plant is the roots. Therefore, the polysaccharide may be isolated from the roots of a plant of the Malvales order. The polysaccharide may be from the roots of a Sida spp. (e.g. Sida cordifolia) or a Malva spp. (e.g. Malva sylvestris) or a Malvastrum spp. (e.g. Malvastrum lateritium) or a Sidalcea spp. (e.g. Sidalcea malviflora). The plant may be the roots of a Sida spp. (e.g. Sida cordifolia) or a Malva spp. (e.g. Malva sylvestris) or a Malvastrum spp. (e.g. Malvastrum lateritium) or a Sidalcea spp. (e.g. Sidalcea malviflora). Most preferably the polysaccharide is isolated from the roots of Sida cordifolia. Thus, the plant may be the roots of Sida cordifolia.

The polysaccharide may be a homopolysaccharide or a heteropolysaccharide. The polysaccharide may be an arabinan polysaccharide. The polysaccharide may be an arabinan homopolysaccharide. The residues of the homopolysaccharide may be L-arabinofuranose residues or be D-arabinofuranose residues. Preferably the residues are L-arabinofuranose residues.

Each repeating unit of the backbone of the polysaccharide referred to within the first aspect may further comprise 3 or more, 4 or more, 5 or more, 6 or more, 7 or more, 8 or more, 9 or more or 10 additional residues (glycosidic) linked to a terminal residue of the backbone. Preferably the additional residues of the backbone are alpha-(1-5)-linked arabinofuranose residues. More preferably the additional residues of the backbone are alpha-L-(1-5)-linked arabinofuranose residues. One or more, two or more, three or more, four or more, five or more, six or more, seven or more, eight or more or nine or more of the additional alpha-(1-5)-linked arabinofuranose residues may or may not comprise any side chains. One or more of the additional alpha-(1-5)-linked arabinofuranose residues may comprise a side chain of a single alpha-(1-2)-linked arabinofuranose residue. One or more of the additional alpha-(1-5)-linked arabinofuranose residues may comprise a side chain of a single alpha-(1-3)-linked arabinofuranose residue. One, two or three of the additional alpha-(1-5)-linked arabinofuranose residues may comprise a side chain of a single alpha-(1-2)-linked arabinofuranose residue and a side chain of a single alpha-arabinofuranose residue (1-3)-linked to the same additional arabinofuranose residue of the backbone.

Each of the repeating units may independently be branched or unbranched. Thus, at least about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95% or about 100% of the repeating units may be branched. Thus, less than about 100%, about 90%, about 95%, about 90%, about 85%, about 80%, about 75%, about 70%, about 65%, about 60%, about 55%, about 50%, about 45%, about 40%, about 35%, about 30%, about 25%, about 20%, about 15%, about 10% or about 5% of the repeating units may be branched.

Each of the repeating units may be directly or indirectly linked to each other. Preferably each of the repeating units is directly linked to each other via a glycosidic bond, such as an alpha-(1-5) glycosidic bond or an alpha-L-(1-5) glycosidic bond.

The first side chain and the second side chain of the polysaccharide according to the invention may be linked to the same arabinofuranose residue of the backbone.

In one embodiment, the repeating unit comprises or consists of Formula (I) (which may also be referred to herein as block “A”), defined herein as follows:

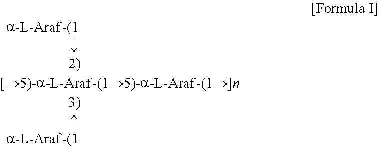

The repeating units of the polysaccharide according to the invention may comprise or consist of blocks referred to herein as blocks “A”, “E” and “F”. Block A (Formula I) is an essential block of the polysaccharide according to the invention. However, the repeating units of the polysaccharide may further comprise block E and/or block F.

Block E may be represented by Formula II, as follows:

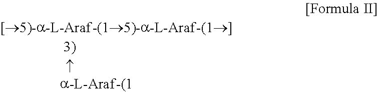

Block F may be represented by Formula III, as follows:

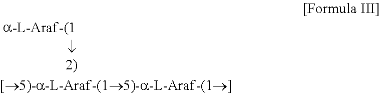

Each block (i.e. block A, block E and block F) comprises a backbone. Each block may be linked by an alpha-(1-5)-glycosidic bond, specifically the backbone of each block. Thus, the repeating units may comprise Formula (I) linked to Formula (II) by an alpha-(1-5)-glycosidic bond. The repeating units may comprise Formula (I) linked to Formula (III) by an alpha-(1-5)-glycosidic bond. The repeating units may comprise Formula (II) linked to Formula (III) by an alpha-(1-5)-glycosidic bond.

Preferably block A is about three, about four or about five times more abundant than block E and/or block F (if E or F is present) in the repeating unit of the polysaccharide.

Thus, the ratio of A:E:F in the repeating units may be 3-5:1:1. Most preferably the ratio of A:E:F is 4:1:1.

Preferably block F (if present in the repeating unit) is the least abundant block of A, E and F. Preferably block A is the most abundant block of A, E and F. Block F (if present in the repeating unit) may be about two, about three, about four, about five, about six, about seven or about less abundant than block A. Block F (if present) may be between about one and about two times less abundant than block A. Preferably for every occurrence of F there is between about 4 and about 6 occurrences of A, and for every occurrence of F there is between about 1 and about 2 occurrences of E. Thus, the ratio of A:E:F in the repeating units may be about 2-8:1-3:1 or 3-7:1-2:1 or 4-6:1-2:1. Most preferably the ratio of A:E:F is 4-6:1-2:1.

Thus, the repeating unit may comprise Formula (IV), defined herein as follows:

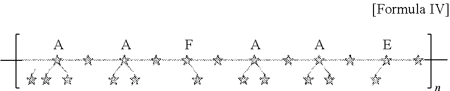

wherein each star of Formula (IV) corresponds to an arabinofuranose, preferably an L-arabinofuranose, more preferably an alpha-arabinofuranose, most preferably an alpha-L-arabinofuranose.

The repeating unit of the polysaccharide may be represented by any one of the combinations shown in Table 1, as follows:

| | | | |
|---|---|---|---|
| AAAAE | AAAAF | AAFAAE | AAEAAF |
| AAAEA | AAAFA | AFAAAE | AEAAAF |
| AAEAA | AAFAA | FAAAAE | EAAAAF |
| AEAAA | AFAAA | AAAFEA | AAAEFA |
| EAAAA | FAAAA | AAFAEA | AAEAFA |
| AAAAEF | AAAAFE | AFAAEA | AEAAFA |
| AAAEFA | AAAFEA | FAAAEA | EAAAFA |
| AAAEFA | AAAFEA | AAFEAA | AAEFAA |
| AAEFAA | AAFEAA | AFAEAA | AEAFAA |
| AEFAAA | AFEAAA | FAAEAA | EAAFAA |
| EFAAAA | FEAAAA | AFEAAA | AEFAAA |
| AAAFAE | AAAEAF | FAEAAA | EAFAAA |

Each cell of Table 1 above represents an embodiment of a repeating unit of the polysaccharide according to the invention. Thus the repeating unit of the polysaccharide may be represented by any one of the 48 examples shown in Table 1. The polysaccharide may comprise “n” repeating units, e.g. “n” repeating units of Formula (I) or Formula (II) or any of the 48 examples shown in Table 1. “n” may be 2 or more, 3 or more, 4 or more, 5 or more 6 or more, 7 or more, 8 or more, 9 or more, 10 or more, 15 or more, 20 or more, 25 or more, 30 or more, 35 or more, or 40 or more, 50 or more, 100 or more, 200 or more, 300 or more, 500 or more, or 1000 or more. “n” may be about 5 to about 1000, about 10 to about 500, or about 15 to about 250, or about 15 to about 230, or about 15 to about 220. Preferably “n” is about 15 to about 220 or about 15 to about 230.

The polysaccharide according to the invention may or may not be a rhamnogalacturonan, such as rhamnogalacturonan-I of rhamnogalacturonan-II.

The polysaccharide or composition referred to herein may be administered several different routes, including, for example, oral, rectal, nasal, pulmonary, topical (including buccal and sublingual), transdermal, intracisternal, intraperitoneal, vaginal and parenteral (including subcutaneous, intramuscular, intrathecal, intravenous and intradermal) route. The polysaccharide or composition referred to herein may be administered orally. The polysaccharide or composition referred to herein may be administered rectally. The polysaccharide or composition referred to herein may be administered nasally. The polysaccharide or composition referred to herein may be administered via the pulmonary route. The polysaccharide or composition referred to herein may be administered topically (e.g. buccally or sublingually). The polysaccharide or composition referred to herein may be administered transdermally. The polysaccharide or composition referred to herein may be administered intracisternally. The polysaccharide or composition referred to herein may be administered intraperitoneally. The polysaccharide or composition referred to herein may be administered via the vaginal. The polysaccharide or composition referred to herein may be administered parenterally (e.g. subcutaneously, intramuscularly, intrathecally, intravenously or intradermally). Preferably the polysaccharide or composition is administered orally.

As mentioned above, a polysaccharide or a composition referred to herein for use according to the invention may be used to treat, prevent and/or ameliorate a neurodegenerative disease and/or symptoms thereof.

A neurodegenerative disease is any medical disease or medical condition that is caused by the death of neurons of the central nervous system (e.g. the brain) and/or the peripheral nervous system. The death of the neurons results in symptoms which may affect speech, movement, memory, intelligence and/or more.

The neurodegenerative disease referred to herein may be a disease selected from the group comprising or consisting of Alzheimer's disease (AD) and/or related dementias, such as vascular dementia, frontotemporal dementia and/or lewy body dementia; Parkinson's disease (PD) and/or PD-related disorders; Huntington's disease (HD); dementia; Motor neurone diseases (MND) (also referred to as amyotrophic lateral sclerosis (ALS)); Spinocerebellar ataxia (SCA); Spinal muscular atrophy (SMA); prion disease; autism spectrum disorders and/or the neurodegenerative process associated with autism spectrum disorders; depression; and schizophrenia. Preferably, the neurodegenerative disease is Alzheimer's disease or Parkinson's disease.

The neurodegenerative disease referred to herein may be one or more diseases selected from the group comprising or consisting of Alzheimer's disease; ALS; and Huntington's disease.

Thus, in one embodiment, there is a polysaccharide comprising “n” repeating units of Formula (I), Formula (IV) or any of the 48 examples shown in Table 1, for use in treating, preventing and/or ameliorating a selection of one or more diseases from the group comprising/consisting of Alzheimer's disease, Parkinson's disease, Huntington's disease, motor neuron disease, prion disease, and/or symptoms thereof in a subject.

In another embodiment, there is a polysaccharide comprising “n” repeating units of Formula (I), Formula (IV) or any of the 48 examples shown in Table 1, for use in treating, preventing and/or ameliorating Alzheimer's disease and/or symptoms thereof in a subject.

In another embodiment, there is a polysaccharide comprising “n” repeating units of Formula (I), Formula (IV) or any of the 48 examples shown in Table 1, for use in treating, preventing and/or ameliorating Parkinson's disease and/or symptoms thereof in a subject.

In another embodiment, there is a polysaccharide comprising “n” repeating units of Formula (I), Formula (IV) or any of the 48 examples shown in Table 1, for use in treating, preventing and/or ameliorating Huntington's disease and/or symptoms thereof in a subject.

In another embodiment, there is a polysaccharide comprising “n” repeating units of Formula (I), Formula (IV) or any of the 48 examples shown in Table 1, for use in treating, preventing and/or ameliorating motor neuron disease and/or symptoms thereof in a subject.

In another embodiment, there is a polysaccharide comprising “n” repeating units of Formula (I), Formula (IV) or any of the 48 examples shown in Table 1, for use in treating, preventing and/or ameliorating prion disease and/or symptoms thereof in a subject.

The inventors believe, but do not wish to be bound by the theory that the neuroinflammatory response is subjects with a neuroinflammatory disorder is hyporesponsive. This hyporesponsiveness is believed to have detrimental effects on the brain and contribute to the progression of neurodegenerative diseases. Given that the polysaccharide according to the invention may be used to enhance neuroinflammation in a subject with a neurodegenerative disorder (see Example 7), the polysaccharide according to the invention may be used to treat a neurodegenerative disorder and/or the symptoms thereof.

Symptoms of Alzheimer's disease include chronic and progressive memory loss (recognition and spatial memory, visuo-spatial dysfunction), disorientation to time and place, impairments in social and occupational functioning (executive function difficulties), deficits in motor function and speech, nominal dysphasia, apathy, personality changes and behavioural and psychological disturbances. Pathological brain changes include beta amyloid plaques, neurofibrillary tangles, marked neuroinflammation and neurodegeneration. A selection of one or more of these symptoms may be treated by a polysaccharide according to the invention.

Symptoms of Parkinson's disease include cardinal features of resting tremor, rigidity, bradykinesia and postural instability. Other common symptoms include masked facies, hypophonia, hypokinetic dysarthria, micrographia, shuffling gait, stooped posture, fatigue, constipation, depression, anxiety, dementia. Pathological brain changes include loss of dopaminergic neurons in the substantia nigra pars compacta (SNc) and accumulation of Lewy bodies (cytoplasmic protein inclusions composed of alpha synuclein protein) as well as mitochondrial dysfunction, neuroinflammation and neurodegeneration. A selection of one or more of these symptoms may be treated by a polysaccharide referred to herein.

Symptoms of Huntington's disease include chorea, incoordination, cognitive decline, personality changes, and psychiatric symptoms, culminating in immobility, mutism, and inanition. Other common symptoms include cognitive dysfunction, impaired social and occupational functioning, irritability and impulsivity, twitching or restlessness, loss of coordination, deficits in motor coordination, impaired concentration, disinhibition or unusually anxious behaviour, depression, obsessions and compulsions. Huntington's disease is caused by an expanded CAG repeat at the N-terminus of the gene that encodes the huntingtin protein, which generates an elongated polyglutamine tail that promotes aggregation. Aggregates interfere with normal cellular functions, such as mitochondrial function, transcriptional regulation, axonal and vesicular transport, apoptosis, proteasome function, and cell-cell interactions. A selection of one or more of these symptoms may be treated by a polysaccharide referred to herein.

Symptoms of motor neuron disease may comprise upper extremity weakness, stiffness with poor coordination and balance, spastic, unsteady gait, painful muscle spasms, difficulties arising from chairs and climbing stairs, foot drop, progressive difficulties in maintaining posture, muscle atrophy, hyper reflexia, dysponea, coughing and choking on liquids and food, strained slow speech, propensity for falls, sialorrhoea and drooling, inappropriate emotional outbursts, cognitive impairment, features of frontotemporal dementia. Pathological mechanisms involved include protein misfolding, glutamate toxicity, oxidative stress, microglial activation, mitochondrial dysfunction, disrupted axonal transport, RNA metabolism dysregulation. A selection of one or more of these symptoms may be treated by a polysaccharide referred to herein.

Symptoms of prion disease may comprise cognitive impairment, ataxia, myoclonus, parkisnsonism, agitation, depression and other psychiatric features, visual changes, insomnia, dysautomia, dizziness and non-specific or constitutional symptoms, sensory symptoms and movement disorders. Pathologically, prion diseases are characterized by presence of pathogenic prion proteins or PrPSc, vacuoles, neutron loss and astrogliosis. A selection of one or more of these symptoms may be treated by a polysaccharide referred to herein.

Symptoms of Lewy body dementia may comprise cognitive fluctuations, visual hallucinations, auditory hallucinations, olfactory hallucinations, tactile hallucinations, movement disorders, parkinsonian symptoms (such as slowed movement, rigid muscles, tremor or shuffling when walking), rapid eye movement (REM) sleep behavioural disturbance, dizziness, poor regulation of body functions (dysregulated blood pressure, pulse, sweating and the digestive processes and constipation), cognitive dysfunction, confusion, poor attention, visual-spatial problems and memory loss, sleep disorders, fluctuating attention, drowsiness, disorganized speech, depression and apathy. Pathologically, lewy body dementia is characterized by the presence of lewy bodies, composed of alpha-synuclein protein. Mitochondrial dysfunction, neuroinflammation and neurodegeneration are also apparent. A selection of one or more of these symptoms may be treated by a polysaccharide referred to herein.

Symptoms of frontotemporal dementia may comprise inappropriate social behaviour, loss of empathy, poor judgment, loss of inhibition, apathy, repetitive compulsive behaviour (such as tapping, clapping or smacking lips), altered eating habits (usually overeating or developing a preference for sweets and carbohydrates), eating inedible objects, speech and language problems (such as primary progressive aphasia, semantic dementia and progressive agrammatic (nonfluent) aphasia), movement-related problems (including tremor, rigidity, muscle spasms, poor coordination, difficulty swallowing, and muscle weakness). Pathologically, frontotemporal dementia is associated with focal degeneration of frontal and temporal lobes, associated with intra-neuronal and glial cell inclusions composed of tau protein or ubiquitin. Mitochondrial dysfunction, neuroinflammation and neurodegeneration are also apparent. A selection of one or more of these symptoms may be treated by a polysaccharide referred to herein.

Symptoms of vascular dementia may comprise difficulties in problem solving, disinhibition, apathy, slowed processing of information, inability to maintain attention, frontal release reflexes, focal neurological signs, impaired balance and gait, cognitive dysfunction, particular difficulties in executive function, motor impairment. Pathologically damage is observed in both grey and white matter from vascular causes: small-vessel changes, infarction, ischaemia and haemorrhage. Mitochondrial dysfunction, neuroinflammation and neurodegeneration are also apparent. A selection of one or more of these symptoms may be treated by a polysaccharide referred to herein.

Symptoms of Autism spectrum disorders include social communication and interaction impairments and restricted, repetitive, and stereotyped patterns of behaviors, interests, or activities, language delays or regression, verbal and non-verbal communication impairments, unusual posturing, motor stereotypes, sensory interests, macrocephaly. Mitochondrial dysfunction, neuroinflammation and neurodegeneration are also apparent. A selection of one or more of these symptoms may be treated by a polysaccharide referred to herein.

Symptoms of depression include low mood, anhedonia, weight changes, sleep disturbance, libido changes, low energy, psychomotor problems, excessive guilt, poor concentration, and suicidal ideation. Mitochondrial dysfunction, neuroinflammation and neurodegeneration are also apparent. A selection of one or more of these symptoms may be treated by a polysaccharide referred to herein.

Symptoms of schizophrenia include delusions, hallucinations, disorganised speech, disorganised/catatonic behaviour, asocial behavior, affective flattening, avolition, anhedonia, attention deficits, alogia, cognitive deficits, somatisation, depression, anxiety, motor coordination deficits. Mitochondrial dysfunction, neuroinflammation and neurodegeneration are also apparent. A selection of one or more of these symptoms may be treated by a polysaccharide referred to herein.

A symptom may be treated and/or ameliorated so that it is equal to or better than that of the average of a population of “m” age- and/or gender-matched subjects without a neurodegenerative disease, or improved compared to the subject's symptom before treatment with the plant or polysaccharide. Treatment of a symptom may be preventing its progression. A symptom may be treated by preventing or reversing its progression within a subject.

As shown, in the Examples, an extract from a plant (i.e. the roots of Sida cordifolia) comprising a polysaccharide of Formula (I) or any one of the 48 examples shown in Table 1 may be used to:
treat/improve impaired memory in a subject with a neurodegenerative disease, such as Alzheimer's disease (e.g. treat/improve impaired memory (such as recognition memory, spatial learning and memory and/or reversal learning and memory) in a subject with a neurodegenerative disease, such as Alzheimer's disease, compared to the average recognition memory, spatial learning and memory and/or reversal learning and memory performance of a population of “m” age- and/or gender matched subjects with the same neurodegenerative disease before treatment with the plant or polysaccharide, or compared to the subject's recognition memory, spatial learning and memory and/or reversal learning and memory performance before treatment with the plant or polysaccharide);
treat or reduce beta amyloid plaque load in the cortex of a subject with a neurodegenerative disease, such as Alzheimer's disease (e.g. reduce beta amyloid plaque load in the cortex of a subject with a neurodegenerative disease, such as Alzheimer's disease, compared to the average beta amyloid plaque load of a population of “m” age- and/or gender matched plant polysaccharide treatment naive subjects with the same neurodegenerative disease before treatment with the plant or polysaccharide, or compared to the average beta amyloid plaque load in the subject before treatment with the plant or polysaccharide);
treat or reduce the levels of astrocytes in the cerebral cortex of a subject with a neurodegenerative disease, such as Alzheimer's disease (e.g. treat or reduce the levels of astrocytes in the cerebral cortex of a subject with a neurodegenerative disease, such as Alzheimer's disease, compared to the levels of astrocytes in the cerebral cortex of the subject before treatment with the plant or polysaccharide, or compared to the average levels of astrocytes in a population of “m” age- and/or gender matched plant polysaccharide treatment naive subjects with the same neurodegenerative disease);
reduce levels of microglia in the cerebral cortex of a subject with a neurodegenerative disease, such as Alzheimer's disease (e.g. reduce levels of microglia in the cerebral cortex of a subject with a neurodegenerative disease, such as Alzheimer's disease, compared to the levels of microglia in the cerebral cortex of the subject before treatment with the plant or polysaccharide, or compared to the average levels of microglia in a population of “m” age- and/or gender matched plant polysaccharide treatment naive subjects with the same neurodegenerative disease); and/or
reduce levels of oxidative stress in the cerebral cortex of a subject with a neurodegenerative disease, such as Alzheimer's disease (e.g. reduce levels of oxidative stress in the cerebral cortex of a subject with a neurodegenerative disease, such as Alzheimer's disease, compared to the level of oxidative stress in the cerebral cortex of the subject before treatment with the plant or polysaccharide, or compared to the average levels of oxidative stress in a population of “m” age- and/or gender matched plant polysaccharide treatment naive subjects with the same neurode generative disease).

“m” may be about 5 or more, about 10 or more, about 20 or more, about 30 or more, about 40 or more, about 50 or more, about 60 or more, about 70 or more, about 80 or more, about 90 or more, about 100 or more, about 110 or more, about 120 or more, about 130 or more, about 140 or more, or about 150 or more subjects.

Pharmaceutical compositions according to the invention may further comprise a pharmaceutically acceptable salt or other form thereof. Pharmaceutical compositions according to the invention may comprise one or more pharmaceutically acceptable excipients, such as carriers, diluents, fillers, disintegrants, lubricating agents, binders, colorants, pigments, stabilizers, preservatives, antioxidants, and/or solubility enhancers. Pharmaceutical compositions according to the invention may comprise a pharmaceutically acceptable salt and optionally one or more pharmaceutically acceptable excipients.

The pharmaceutical compositions can be formulated by techniques known in the art. The pharmaceutical compositions can be formulated as dosage forms for oral, parenteral, such as topical, transdermal, intramuscular, intravenous, subcutaneous, intradermal, intraarterial, intracardial, nasal or aerosol administration. The pharmaceutical composition may be formulated as a dosage form for oral administration.

In the present context, the term “pharmaceutically acceptable salt” is intended to indicate salts which are not harmful to a patient. Such salts include pharmaceutically acceptable acid addition salts, pharmaceutically acceptable metal salts, ammonium and alkylated ammonium salts. Acid addition salts include salts of inorganic acids as well as organic acids. Representative examples of suitable inorganic acids include hydrochloric, hydrobromic, hydroiodic, phosphoric, sulfuric, nitric acids and the like. Representative examples of suitable organic acids include formic, acetic, trichloroacetic, trifluoroacetic, propionic, benzoic, cinnamic, citric, fumaric, glycolic, lactic, maleic, malic, malonic, mandelic, oxalic, picric, pyruvic, salicylic, succinic, methanesulfonic, ethanesulfonic, tartaric, ascorbic, pamoic, bismethylene salicylic, ethanedisulfonic, gluconic, citraconic, aspartic, stearic, palmitic, EDTA, glycolic, p-aminobenzoic, glutamic, benzenesulfonic, p-toluenesulfonic acids and the like. Further examples of pharmaceutically acceptable inorganic or organic acid addition salts include the pharmaceutically acceptable salts listed in J. Pharm. Sci. 1977, volume 66, issue 2. Examples of metal salts include lithium, sodium, potassium, magnesium salts and the like. Examples of ammonium and alkylated ammonium salts include ammonium, methylammonium, dimethylammonium, trimethylammonium, ethylammonium, hydroxyethylammonium, diethylammonium, butylammonium, tetramethylammonium salts and the like.

The pharmaceutical composition according to the invention may be formulated with pharmaceutically acceptable carriers or diluents as well as any other known adjuvants and excipients in accordance with conventional techniques such as those disclosed in Remington: The Science and Practice of Pharmacy, 19th Edition, Gennaro, Ed., Mack Publishing Co., Easton, PA, 1995.

Suitable pharmaceutical carriers include inert solid diluents or fillers, sterile aqueous solutions and various organic solvents. Examples of solid carriers are lactose, terra alba, sucrose, cyclodextrin, talc, gelatine, agar, pectin, acacia, magnesium stearate, stearic acid and lower alkyl ethers of cellulose. Examples of liquid carriers are syrup, peanut oil, olive oil, phospholipids, fatty acids, fatty acid amines, polyoxyethylene and water. In addition, the compounds of the invention may form solvates with water or common organic solvents. Such solvates are also encompassed within the scope of the present invention.

The composition may further comprise a buffer system, preservative(s), tonicity agent(s), chelating agent(s), stabilizers and surfactants, which is well known to the skilled person. For convenience reference is made to Remington: The Science and Practice of Pharmacy, 20th edition, 2000. The composition may also further comprise one or more therapeutic agents active against the same disease state.

Methods to produce controlled release systems useful for compositions of the current invention include, but are not limited to, crystallization, condensation, co-crystallization, precipitation, co-precipitation, emulsification, dispersion, high pressure homogenisation, encapsulation, spray drying, microencapsulating, coacervation, phase separation, solvent evaporation to produce microspheres, extrusion and supercritical fluid processes. General reference is made to Handbook of Pharmaceutical Controlled Release (Wise, D. L., ed. Marcel Dekker, New York, 2000) and Drug and the Pharmaceutical Sciences vol. 99: Protein Composition and Delivery (MacNally, E. J., ed. Marcel Dekker, New York, 2000).

Administration of pharmaceutical compositions according to the invention may be through several routes of administration, for example, oral, rectal, nasal, pulmonary, topical (including buccal and sublingual), transdermal, intracisternal, intraperitoneal, vaginal and parenteral (including subcutaneous, intramuscular, intrathecal, intravenous and intradermal) route. It will be appreciated that the preferred route will depend on the general condition and age of the subject to be treated, the nature of the condition to be treated and the active ingredient chosen.

For topical use, sprays, creams, ointments, jellies, gels, inhalants, dermal patches, implants, solutions of suspensions, etc., containing the compounds of the present invention are contemplated. For the purpose of this application, topical applications shall include mouth washes and gargles. Compounds of the invention may be used in wafer technology, wafer technology, such as the biodegradable Gliadel polymer wafer, is useful for brain cancer chemotherapy.

Pharmaceutical compositions for oral administration include solid dosage forms such as hard or soft capsules, tablets, troches, dragees, pills, lozenges, powders and granules and liquid dosage forms for oral administration include solutions, emulsions, aqueous or oily suspensions, syrups and elixirs, each containing a predetermined amount of the active ingredient, and which may include a suitable excipient.

Compositions intended for oral use may be prepared according to any known method, and such compositions may contain one or more agents selected from the group consisting of sweetening agents, flavouring agents, colouring agents, and preserving agents in order to provide pharmaceutically elegant and palatable preparations.

Tablets may contain the active ingredient in admixture with non-toxic pharmaceutically-acceptable excipients which are suitable for the manufacture of tablets. These excipients may be for example, inert diluents, such as calcium carbonate, sodium carbonate, lactose, calcium phosphate or sodium phosphate; granulating and disintegrating agents, for example corn starch or alginic acid; binding agents, for example, starch, gelatin or acacia; and lubricating agents, for example magnesium stearate, stearic acid or talc.

The tablets may be uncoated or they may be coated by known techniques to delay disintegration and absorption in the gastrointestinal tract and thereby provide a sustained action over a longer period. For example, a time delay material such as glyceryl monostearate or glyceryl distearate may be employed. They may also be coated by the techniques described in U.S. Pat. Nos. 4,356, 108; 4, 166,452; and 4,265,874 to form osmotic therapeutic tablets for controlled release.

Formulations for oral use may also be presented as hard gelatine capsules where the active ingredient is mixed with an inert solid diluent, for example, calcium carbonate, calcium phosphate or kaolin, or as soft gelatine capsules wherein the active ingredient is mixed with water or an oil medium, for example peanut oil, liquid paraffin, or olive oil.

Aqueous suspensions may contain the active compounds in admixture with excipients suitable for the manufacture of aqueous suspensions. Such excipients are suspending agents, for example sodium carboxymethylcellulose, methylcellulose, hydroxypropylmethylcellulose, sodium alginate, polyvinylpyrrolidone, gum tragacanth and gum acacia; dispersing or wetting agents may be a naturally-occurring phosphatide such as lecithin, or condensation products of an alkylene oxide with fatty acids, for example polyoxyethylene stearate, or condensation products of ethylene oxide with long chain aliphatic alcohols, for example, heptadecaethyl-eneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more colouring agents, one or more flavouring agents, and one or more sweetening agents, such as sucrose or saccharin.

Oily suspensions may be formulated by suspending the active ingredient in a vegetable oil, for example arachis oil, olive oil, sesame oil or coconut oil, or in a mineral oil such as a liquid paraffin. The oily suspensions may contain a thickening agent, for example beeswax, hard paraffin or cetyl alcohol. Sweetening agents such as those set forth above, and flavouring agents may be added to provide a palatable oral preparation. These compositions may be preserved by the addition of an anti-oxidant such as ascorbic acid.

Dispersible powders and granules suitable for preparation of an aqueous suspension by the addition of water provide the active compound in admixture with a dispersing or wetting agent, suspending agent and one or more preservatives. Suitable dispersing or wetting agents and suspending agents are exemplified by those already mentioned above. Additional excipients, for example, sweetening, flavouring, and colouring agents may also be present.

The pharmaceutical compositions of the present invention may also be in the form of oil-in-water emulsions. The oily phase may be a vegetable oil, for example, olive oil or arachis oil, or a mineral oil, for example a liquid paraffin, or a mixture thereof. Suitable emulsifying agents may be naturally-occurring gums, for example gum acacia or gum tragacanth, naturally-occurring phosphatides, for example soy bean, lecithin, and esters or partial esters derived from fatty acids and hexitol anhydrides, for example sorbitan monooleate, and condensation products of said partial esters with ethylene oxide, for example polyoxyethylene sorbitan monooleate. The emulsions may also contain sweetening and flavouring agents.

Syrups and elixirs may be formulated with sweetening agents, for example glycerol, propylene glycol, sorbitol or sucrose. Such formulations may also contain a demulcent, a preservative, a flavouring agent and a colouring agent. The pharmaceutical composition may be in the form of a sterile injectable aqueous or oleaginous suspension. This suspension may be formulated according to known methods using suitable dispersing or wetting agents and suspending agents described above. The sterile injectable preparation may also be a sterile injectable solution or suspension in a non-toxic parenterally-acceptable diluent or solvent, for example as a solution in 1,3-butanediol. Among the acceptable vehicles and solvents that may be employed are water, Ringer's solution, and isotonic sodium chloride solution. In addition, sterile, fixed oils are conveniently employed as a solvent or suspending medium. For this purpose, any bland fixed oil may be employed using synthetic mono- or diglycerides. In addition, fatty acids such as oleic acid find use in the preparation of injectables.

Parenteral administration may be performed by subcutaneous, intramuscular, intraperitoneal or intravenous injection by means of a syringe, optionally a pen-like syringe. Alternatively, parenteral administration can be performed by means of an infusion pump. A further option is a composition which may be a solution or suspension for the administration of the prolactin receptor antagonist in the form of a nasal or pulmonal spray. As a still further option, the pharmaceutical compositions containing the compound of the invention can also be adapted to transdermal administration, e.g. by needle-free injection or from a patch, optionally an iontophoretic patch, or transmucosal, e.g. buccal, administration.

Pharmaceutical compositions for parenteral administration include sterile aqueous and non-aqueous injectable solutions, dispersions, suspensions or emulsions as well as sterile powders to be reconstituted in sterile injectable solutions or dispersions prior to use.

A medicament includes but is not limited to a composition, such as an composition (e.g. a pharmaceutical composition or an edible composition), a prescription drug, a non-prescription drug, an over the counter medicine, a dietary supplement, a dietary food, a clinical food, an edible product, a tablet, a capsule, a pill, and food products such as beverages or any other suitable food product, and any other composition which is commonly known to the skilled person. Alternatively, the medicament may be an injectable substance or an inhalable substance, such as a nasal spray.

The polysaccharide may be added to an edible composition or pharmaceutical composition in a specific salt form. The edible composition according to the present invention may take any physical form. In particular, it may be a food product, a beverage, a dietary food product, or a clinical food product. It may also be a dietary supplement, in the form of a beverage, a tablet, a capsule, a liquid (e.g. a soup or a beverage, a spread, a dressing or a dessert) or any other suitable form for a dietary supplement. The edible composition may be in a liquid or a spreadable form, it may be a spoonable solid or soft-solid product, or it may be a food supplement. Preferably the edible composition is a liquid product. The edible composition may suitably take the form of e.g. a soup, a beverage, a spread, a dressing, a dessert, a bread. The term “spread” as used herein encompasses spreadable products such as margarine, light margarine, spreadable cheese based products, processed cheese, dairy spreads, and dairy- alternative spreads. Spreads as used herein (oil-in-water or water-in-oil emulsions) may have a concentration of oil and/or fat of between about 5% and 85% by weight, preferably between 10% and 80% by weight, more preferred between 20% and 70% by weight. Preferably the oil and/or fat are from vegetable origin (such as but not limited to sunflower oil, palm oil, rapeseed oil); oils and/or fats of non-vegetable origin may be included in the composition as well (such as but not limited to dairy fats, fish oil).

The term “aqueous composition” is defined as a composition comprising at least 50% w/w water. Likewise, the term “aqueous solution” is defined as a solution comprising at least 50% w/w water, and the term “aqueous suspension” is defined as a suspension comprising at least 50% w/w water. Such aqueous solutions should be suitably buffered if necessary and the liquid diluent first rendered isotonic with sufficient saline or glucose. The aqueous solutions are particularly suitable for intravenous, intramuscular, subcutaneous and intraperitoneal administration. The sterile aqueous media employed are all readily available by standard techniques known to those skilled in the art. Depot injectable formulations are also contemplated as being within the scope of the present invention.

The isolated polysaccharide, or the composition may be administered through several routes of administration, for example, oral, rectal, nasal, pulmonary, topical (including buccal and sublingual), transdermal, intracisternal, intraperitoneal, vaginal and parenteral (including subcutaneous, intramuscular, intrathecal, intravenous and intradermal) route. It will be appreciated that the preferred route will depend on the general condition and age of the subject to be treated, the nature of the condition to be treated and the active ingredient chosen. Preferably the isolated polysaccharide or fragment thereof, or the composition is administered orally.

Abbreviations: kDa—kilo Dalton; Gal—D-Galactose; GalA—D-Galacturonic acid; Rha—L-Rhamnose; Ara—L-Arabinose; Fuc—L-Fucose; Glc—D-Glucose; GlcA—D-Glucuronic acid. The L- and D- forms of these monomers and the corresponding polymers (e.g. polysaccharides) as indicated here also apply to the monomers and polymers (e.g. polysaccharides) as indicated in the rest of this specification (which may not be abbreviated but written in full).

Furthermore, the skilled person will appreciate that each star of Formula (I) and Formula (II) corresponds to an arabinofuranose, preferably an L-arabinofuranose, more preferably an alpha-arabinofuranose, most preferably an alpha-L-arabinofuranose.

The term “average” may be the arithmetic mean, the mode or the median. Preferably the average refers to the arithmetic mean.

The term “isolated” can refer to a polysaccharide that is no longer in its natural environment. Thus, the term “isolated” can refer to a polysaccharide that has been separated from Malvaceae/Malvoideae/Malveae plant tissue and cells (such as Sida cordifolia tissue and Sida cordifolia cells).

A “subject” may be a vertebrate, a mammal, a non-human animal or a domestic animal. Hence, polysaccharide, the adjuvant, the vaccine, the composition and the medicament according to the invention may be used to treat any mammal, for example livestock (e.g. a horse), pets, or maybe used in other veterinary applications. Livestock may be bovine, cow, cattle, sheep, horse, chicken, goat, pig, calf, deer, goose, turkey or rabbit. A domestic animal may be a dog or a cat. Preferably the subject is a mammal. Most preferably the mammal is a human being. An organism can refer to a subject.

It will be appreciated that the term “treatment” and “treating” as used herein means the management and care of a subject for the purpose of combating a condition, such as a disease or a disorder. The term is intended to include the full spectrum of treatments for a given condition from which the subject is suffering, including alleviating symptoms or complications, delaying the progression of the disease, disorder or condition, alleviating or relieving the symptoms and complications, and/or to cure or eliminating the disease, disorder or condition as well as to prevent the condition, wherein prevention is to be understood as the management and care of a subject for the purpose of combating the disease, condition, or disorder and includes the administration of the ligand to prevent the onset of the symptoms or complications.

The term “comprising” can refer to “consisting of” or “consisting essentially of”.

All of the embodiments and features described herein (including any accompanying claims, abstract and drawings), and/or all of the steps of any method or process so disclosed, may be combined with any of the above aspects or embodiments in any combination, unless stated otherwise with reference to a specific combinations, for example, combinations where at least some of such features and/or steps are mutually exclusive.

For a better understanding of the invention, and to show embodiments of the invention may be put into effect, reference will now be made, by way of example, to the accompanying drawings, in which:

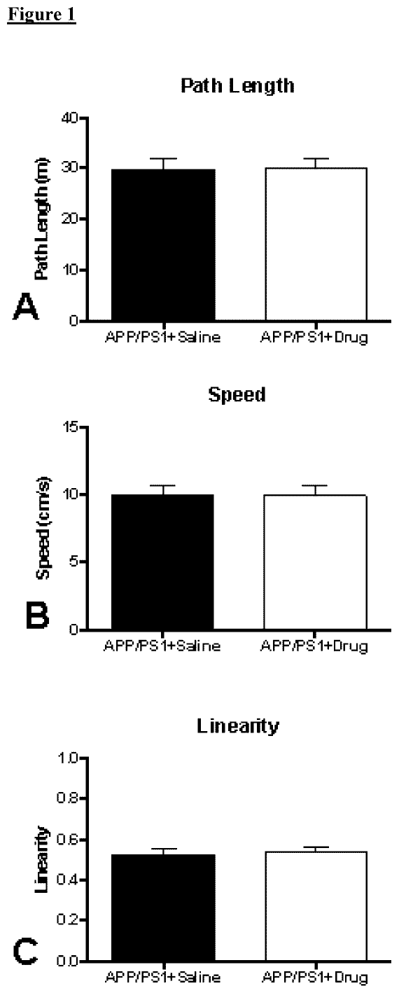

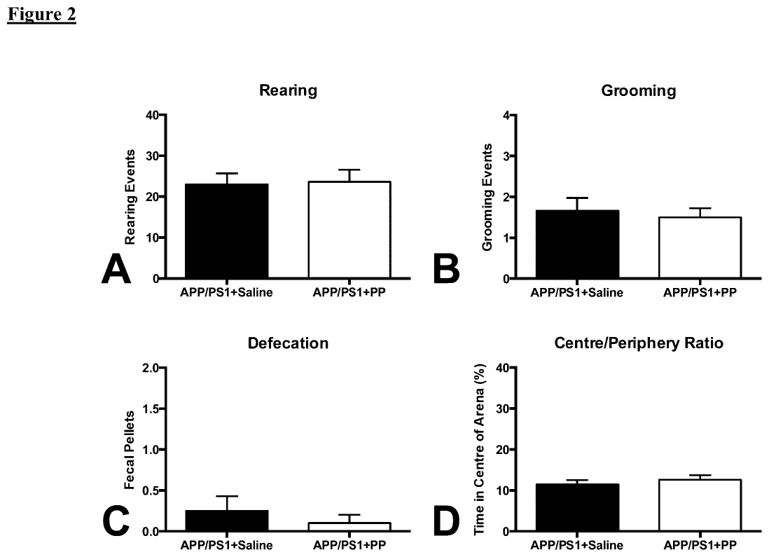

FIG. 1 is a bar graph showing the effect of the PP on the locomotor function of APP/PS1 mice. APP/PS1 and wild-type mice were subjected to assessment in an open field task, which measured path length (A), speed (B) and linearity (C) as measures of locomotor function. Data represent mean ±SEM for 10-12 mice.

FIG. 2 is a bar graph showing the effect of the PP on the anxiety levels of APP/PS1 mice. APP/PSI and wild-type mice aged 7 months were subjected to assessment in an open field task, which measured rearing (A), grooming (B), defecation (C) and time spent in the centre of the arena (D) as measures of anxiety. Data represent mean ±SEM for 10-12 mice.

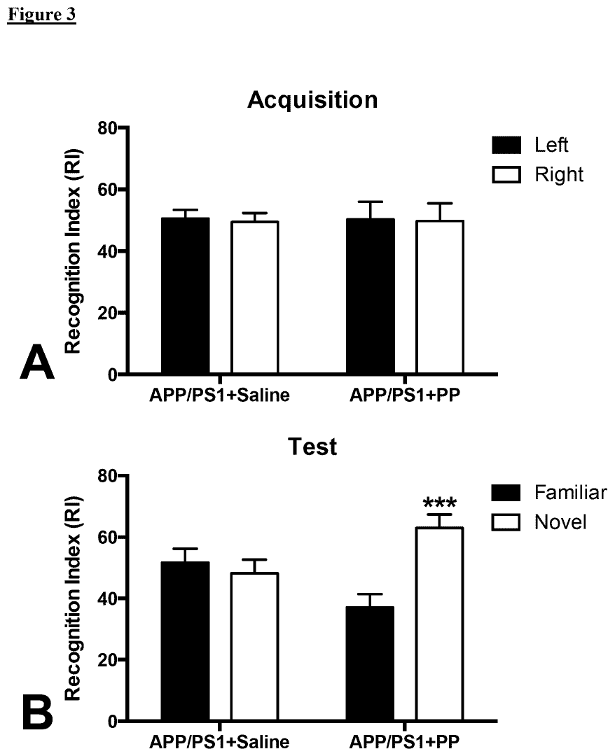

FIG. 3 is a bar graph showing that impaired recognition memory in APP/PS1 mice is restored by PP treatment. Twenty-four hours following exposure to the open field arena, a novel object recognition task (ORT) was conducted using the same arena. Recognition index, a measure of the percentage of time spent exploring either object, is illustrated in the acquisition phase (A) during exposure to two identical objects, and the test phase (B), in the presence of one familiar and one novel object. ***p<0.001, multiple t-tests with Holm-Šídák's multiple comparisons post-hoc test. Data represent mean±SEM for 10-12 mice.

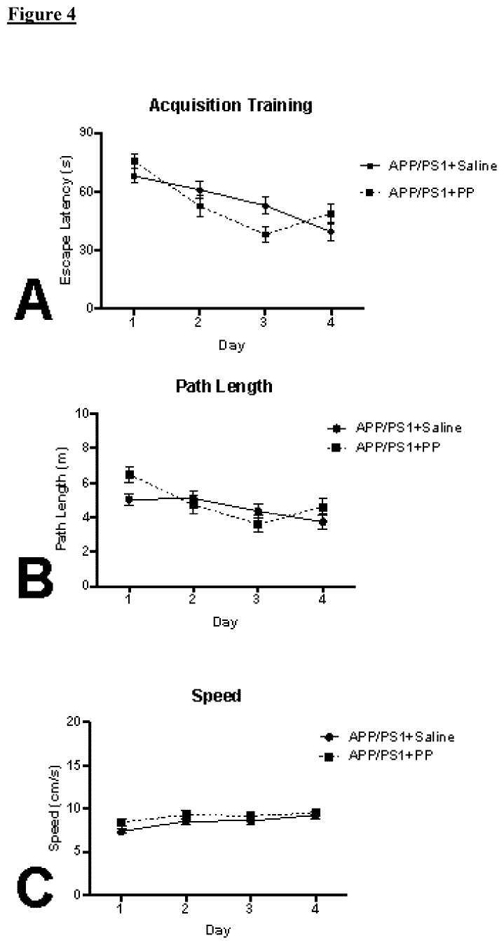

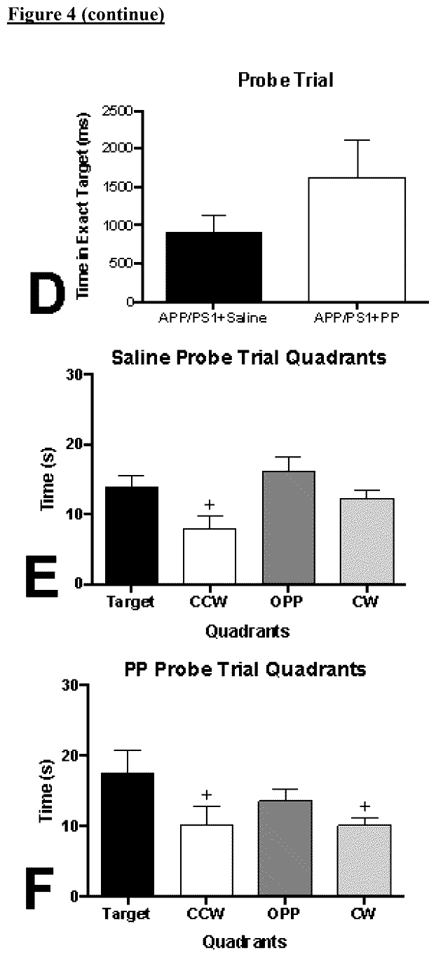

FIG. 4 shows that spatial learning and memory are improved by PP treatment in APP/PS1 mice. The acquisition training phase involved four training sessions per day over four consecutive days. Illustrated are escape latency (A), path length (B) and swim speed (C) for APP/PS1 mice treated with saline or PP during acquisition training. A probe trial was conducted on the fifth day, 24 hours following the final training session as a measure of spatial memory. Time spent in the exact target area is shown (D), as is the proportion of time spent in each quadrant during the probe trial by saline-treated (E) and PP-treated (F) APP/PSI mice. *p<0.05, two-way repeated measures ANOVA with Bonferroni's post-hoc test (A-C), Student's t test (D), p<0.05 vs. target, ordinary one-way ANOVA with Dunnett's post-hoc test (E, F). Data represent mean ±SEM for 10-12 mice per group.

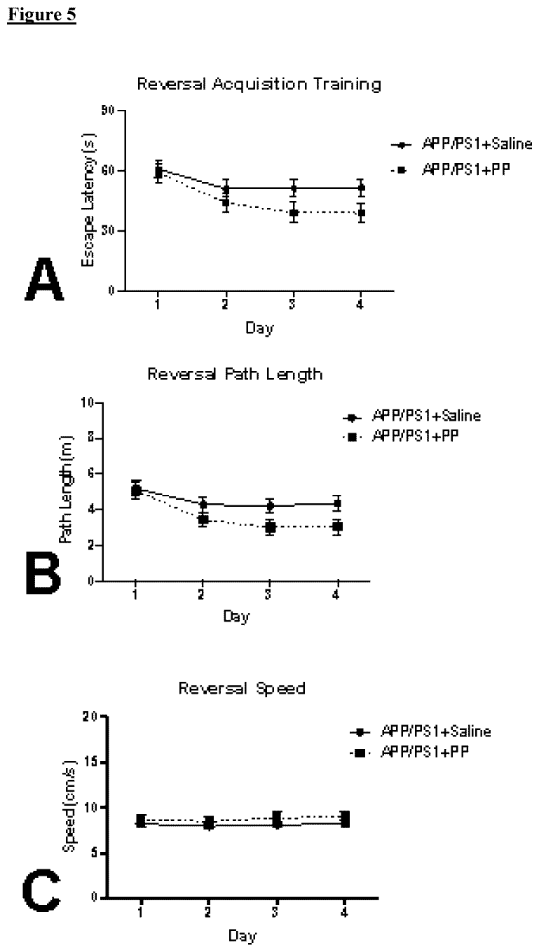

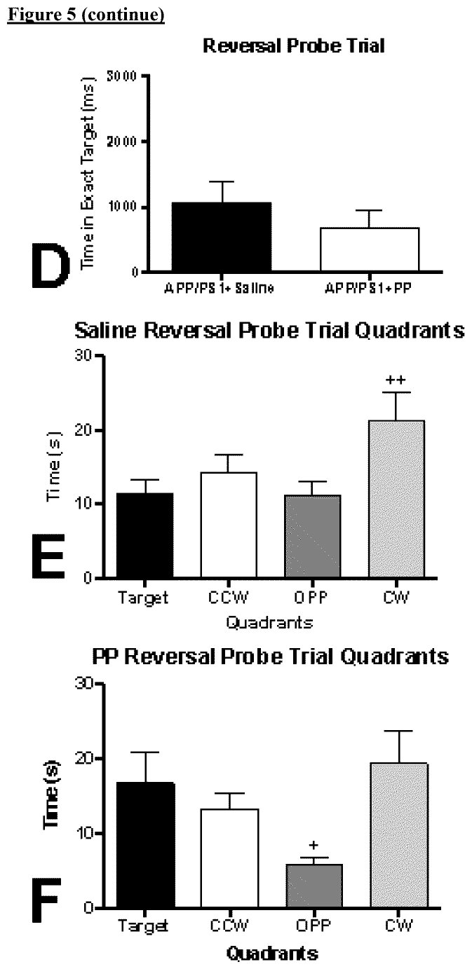

FIG. 5 shows that reversal learning is improved by PP treatment in APP/PSI mice. Reversal water maze training began 24 hours following the Morris water maze probe trial and consisted of four consecutive days with four training sessions per day. Illustrated are escape latency (A), path length (B) and swim speed (C) for APP/PSI treated with saline or PP during reversal acquisition training. A probe trial was conducted on the fifth day, 24 hours following the final training session as a measure of reversal spatial memory. Time spent in the exact target area is shown (D), as is the proportion of time spent in each quadrant during the reversal probe trial by saline-treated (E) and PP-treated (F) APP/PS1 mice. Two-way repeated measures ANOVA with Bonferroni's post-hoc test (A-C), Student's t test (D), +p<0.05, ++p<0.01 vs. target, ordinary one-way ANOVA with Dunnett's post-hoc test (E, F). Data represent mean±SEM for 10-12 mice per group.

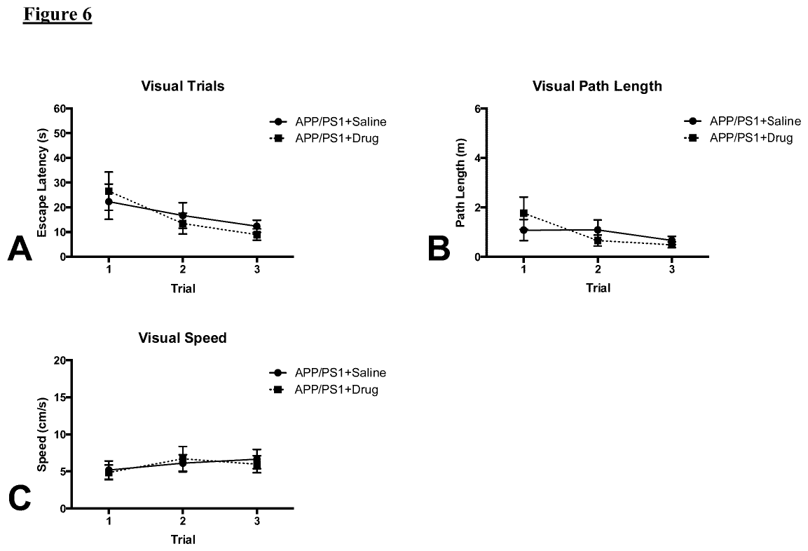

FIG. 6 shows that sensorimotor function in APP/PS1 mice is unaffected by PP treatment. Subsequent to the reversal water maze probe trial, sensorimotor function was assessed in saline-treated and PP-treated APP/PS1 mice in 3 visual water maze trials. Illustrated are escape latency (A), path length (B) and swim speed (C). Data represent mean±SEM for 10-12 mice per group.

FIG. 7 shows that PP treatment reduces AP deposition in the brains of APP/PS1 mice. Representative images (10× magnification) are shown that depict the cerebral cortex (A) and dentate gyrus (D) of APP/PS1 mice treated with saline and the cerebral cortex (B) and dentate gyrus (E) of PP-treated APP/PS1 mice. Also shown in the upper right corner of each image, is an exemplary magnified image (20× magnification). Quantification of AP immunopositivity in the cortex (C) and dentate gyrus (F) of saline-and PP-treated APP/PS1 mice is also shown. ***p<0.001, Student's t-test. Data represent mean±SEM for 6 per group.

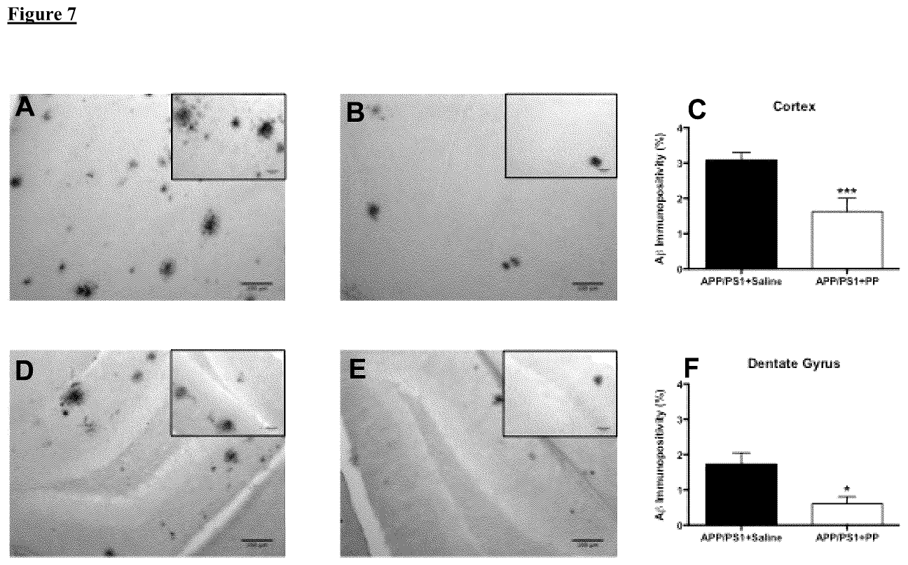

FIG. 8 shows that PP treatment reduces levels of astrocytes in the cerebral cortex of APP/PS1 mice. Representative images (10× magnification) are shown that depict the cerebral cortex (A) and dentate gyrus (D) of APP/PS1 mice treated with saline and the cerebral cortex (B) and dentate gyrus (E) of PP-treated APP/PS1 mice. Also illustrated are exemplary magnified images (20× magnification) of GFAP staining in the cortex of saline- (G) and PP- (H) treated APP/PS1 mice, as well as representative 40× images in the upper right corner (G, H). Quantification of GFAP immunopositivity in the cortex (C) and dentate gyrus (F) of saline- and PP-treated APP/PS1 mice is also shown. *p<0.05, Student's t-test. Data represent mean±SEM for 6 per group.

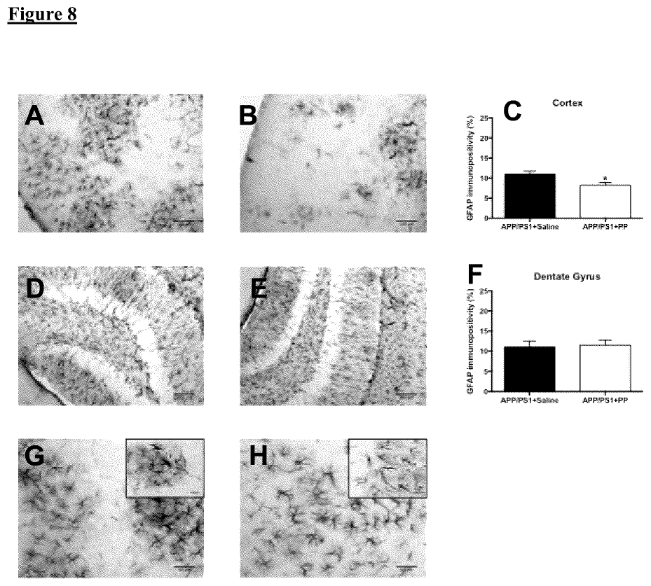

FIG. 9 shows that PP treatment reduces levels of microglia in the cerebral cortex of APP/PS1 mice. Representative images (10× magnification) are shown that depict the cerebral cortex (A) and dentate gyrus (D) of APP/PS1 mice treated with saline and the cerebral cortex (B) and dentate gyrus (E) of PP-treated APP/PS1 mice. Also illustrated are exemplary magnified images (20×) of Ibal staining in the cortex of saline- (G) and PP- (H) treated APP/PS1 mice, as well as representative 40× images in the upper right corner (G, H). Quantification of Ibal immunopositivity in the cortex (C) and dentate gyrus (F) of saline- and PP-treated APP/PS1 mice is also shown. *p<0.05, Student's t-test. Data represent mean±SEM for 6 per group.

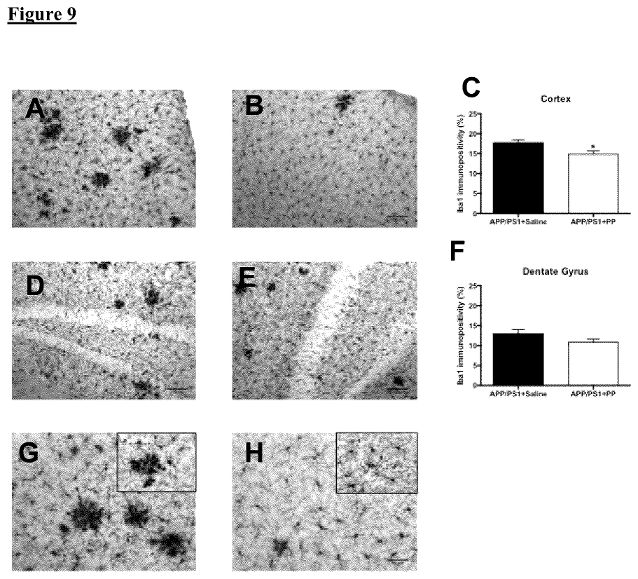

FIG. 10 shows that PP treatment reduces levels of oxidative stress in the cerebral cortex of APP/PS1 mice. Representative images (10× magnification) are shown that depict the cerebral cortex (A) and dentate gyrus (D) of APP/PS1 mice treated with saline and the cerebral cortex (B) and dentate gyrus (E) of PP-treated APP/PS1 mice. Also illustrated are exemplary magnified images (20× magnification) of Ibal staining in the cortex of saline- (G) and PP- (H) treated APP/PS1 mice, as well as representative 40× images in the upper right corner (G, H). *p<0.05, Student's t-test. Data represent mean±SEM for 6 per group.

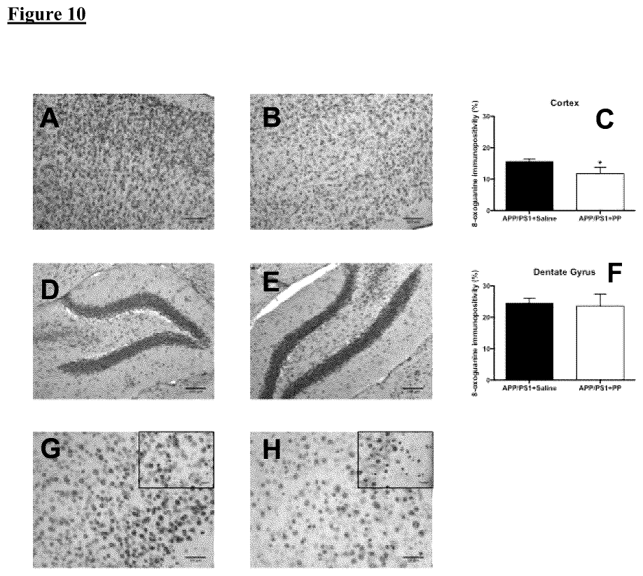

FIG. 11 shows that PP treatment has no effect on levels of IRS-1 pSer 616 in the brains of APP/PS1 mice. Representative images (10× magnification) are shown that depict the cerebral cortex (A) and dentate gyrus (D) of APP/PS1 mice treated with saline and the cerebral cortex (B) and dentate gyrus (E) of PP-treated APP/PS1 mice. Also illustrated are exemplary magnified images (20×) of Ibal staining in the cortex of saline- (G) and PP- (H) treated APP/PS1 mice, as well as representative 40× images in the upper right corner (G, H). Data represent mean±SEM for 6 per group.

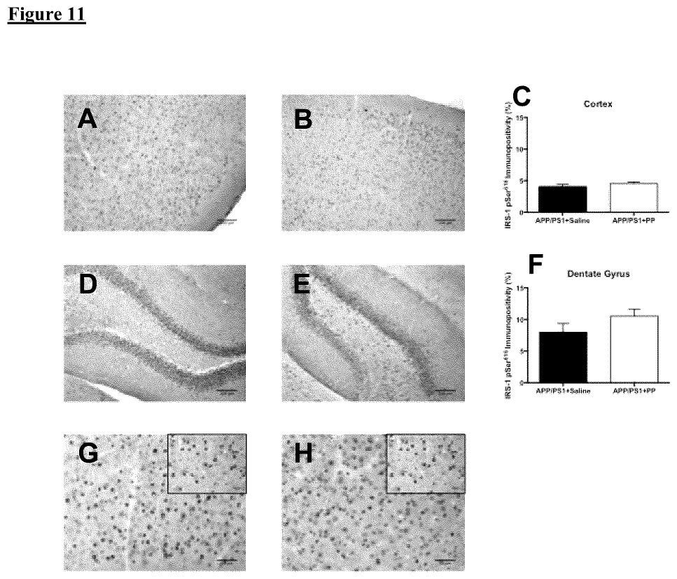

FIG. 12 is a bar graph showing that PP treatment has no effect on spleen weight in APP/PS1 mice. Average weights of spleens from PP-treated APP/PS1 mice are illustrated (A). Data represent mean±SEM for 6 per group.

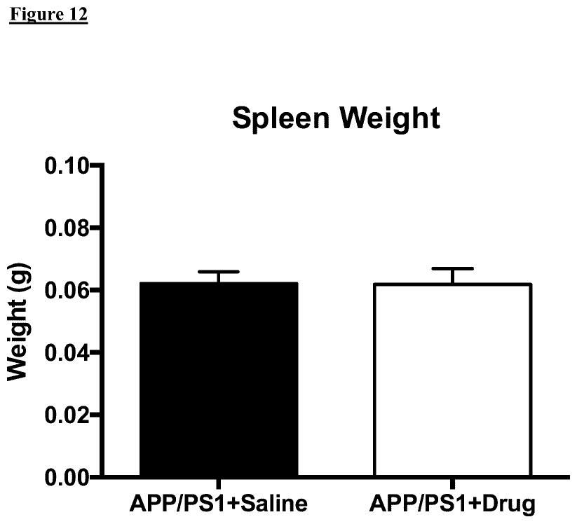

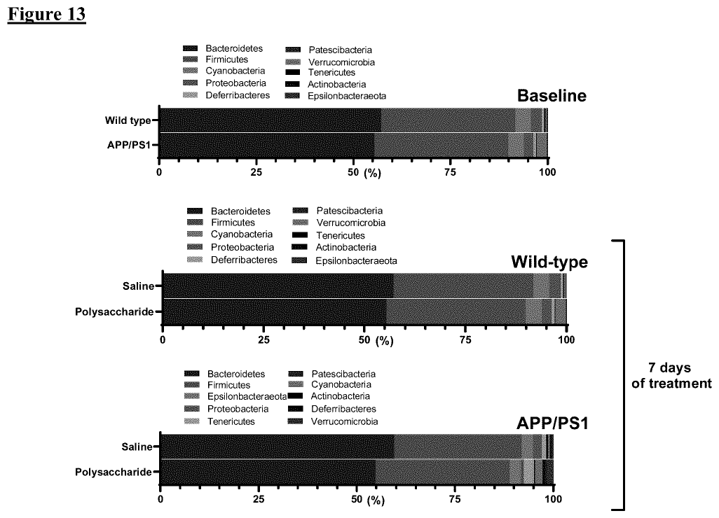

FIG. 13 shows, at the phyla level, the effect of the effect of an isolated polysaccharide according to the invention on the faecal bacterial biodiversity of wild-type and APP/PS1 mice.

FIG. 14 shows the effect of the effect of an isolated polysaccharide according to the invention on the faecal bacterial biodiversity of wild-type and APP/PS1 mice at the phyla and species level.

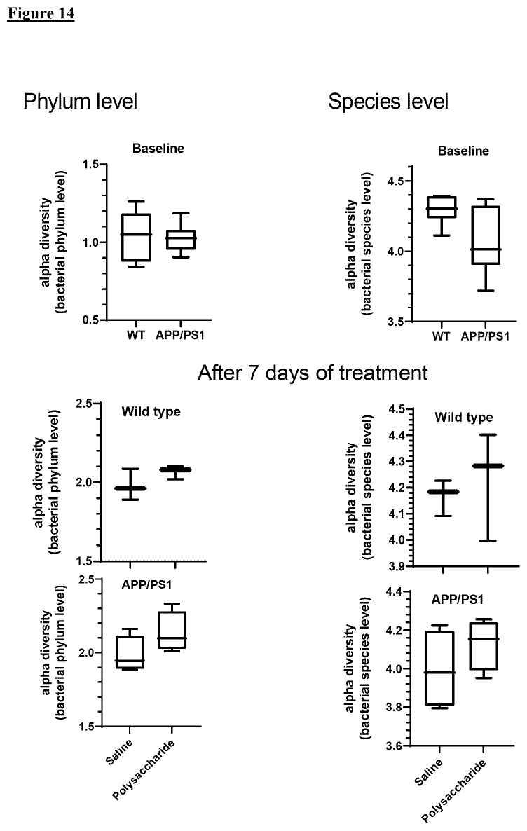

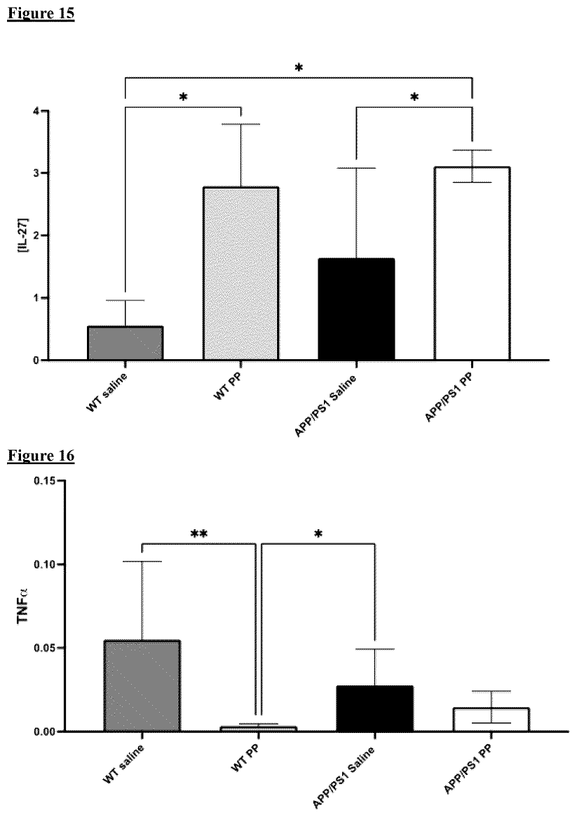

FIG. 15 shows the plasma IL-27 concentration in APP and wild-type mice treated with saline and the polysaccharide (PP) according to the invention for 7 days.

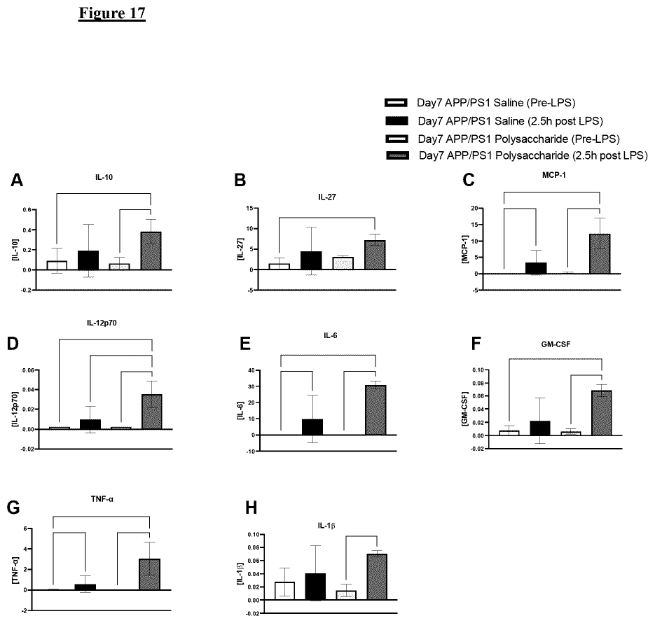

FIG. 16 shows the plasma TNF alpha concentration in APP and wild-type mice treated with saline and the polysaccharide (PP) according to the invention for 7 days.

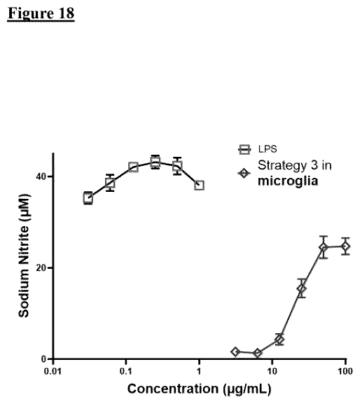

FIG. 17 shows that the polysaccharide potentiates cytokine response after an LPS challenge in APP/PS1 mice.

FIG. 18 is a concentration-effect curve in SIM-A9 microglia treated with the polysaccharide according to the invention or LPS. The measured effect is NO production.

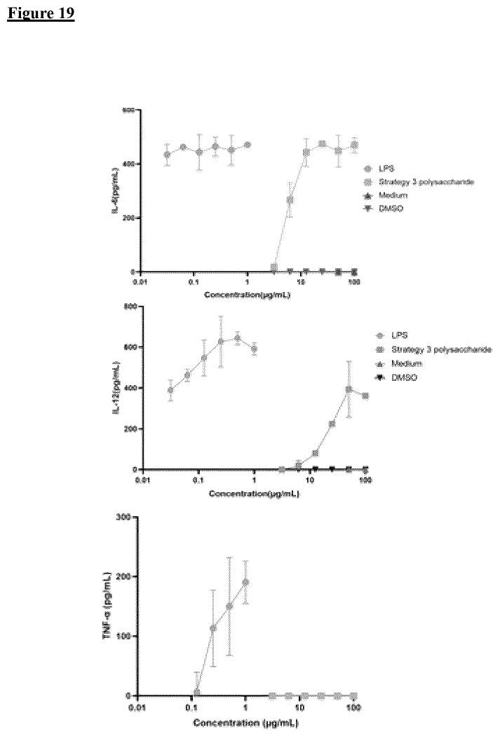

FIG. 19 shows the effect of the polysaccharide according to the invention on cytokine production by microglia.

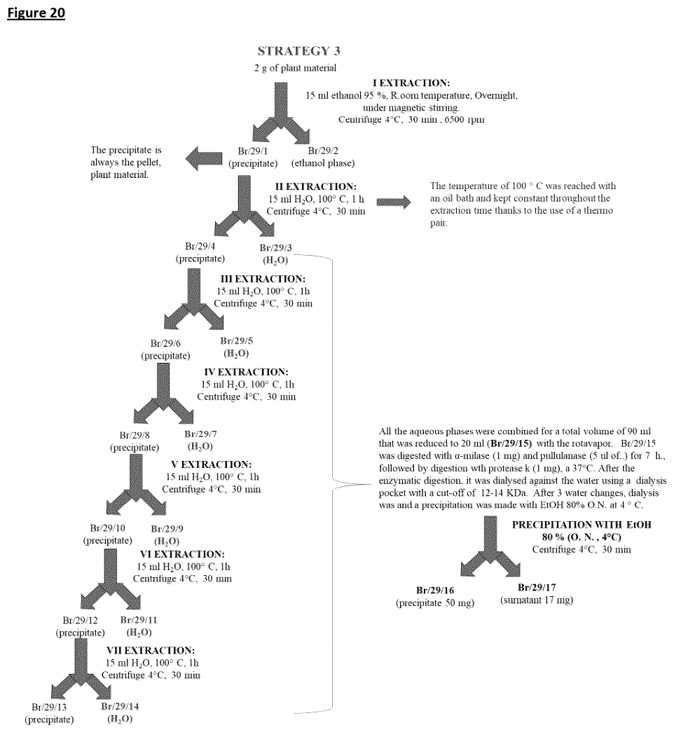

FIG. 20 is a schematic diagram of an optimised extraction protocol for obtaining the active arabinan polysaccharide from plant materials. The flow chart illustrates the 3 main steps required for isolating the polysaccharide including the methodological details. Briefly, the method comprises of a first step involving the use of ethanol to initially extract the polysaccharide followed by a second step involving up to 6 cycles of water (aqueous) extraction, followed by a final enzymatic digestion, dialysis and precipitation step.

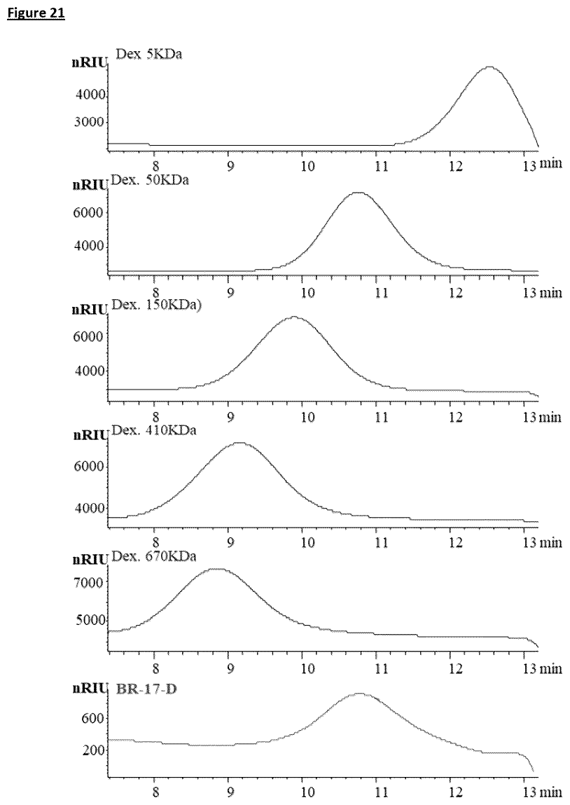

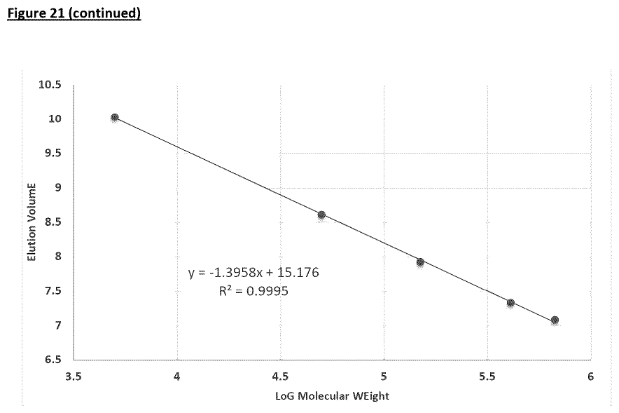

FIG. 21 is an elution profile on Size-Exclusion Chromatography (SEC)-HPLC of dextran at known M.W (in blue) and the arbinan Br/17/D (in red). The comparison with known molecular weight dextrans revealed that the molecular weight of Br/17/D is 49 KDa. Below is an example of a calibration curve based on dextran at known M.W and a typical example of a straight line equation.

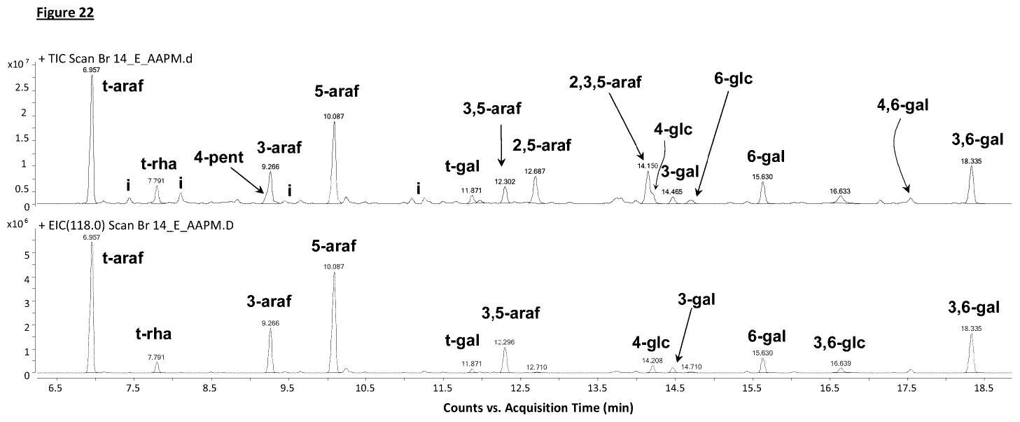

FIG. 22 is a linkage analysis of Br/14/E (less pure fraction). GC-MS analysis of partially methylated alditol acetates (PMAA) of the fraction Br/14/E. The top trace shows the chromatogram referred to the abundance of the total ions. The bottom trace (in red): is shown the chromatogram referred to the abundance of the 118 ion (extract chromatogram ion 118), consequently only the peaks with this ion are showed. I=impurity.

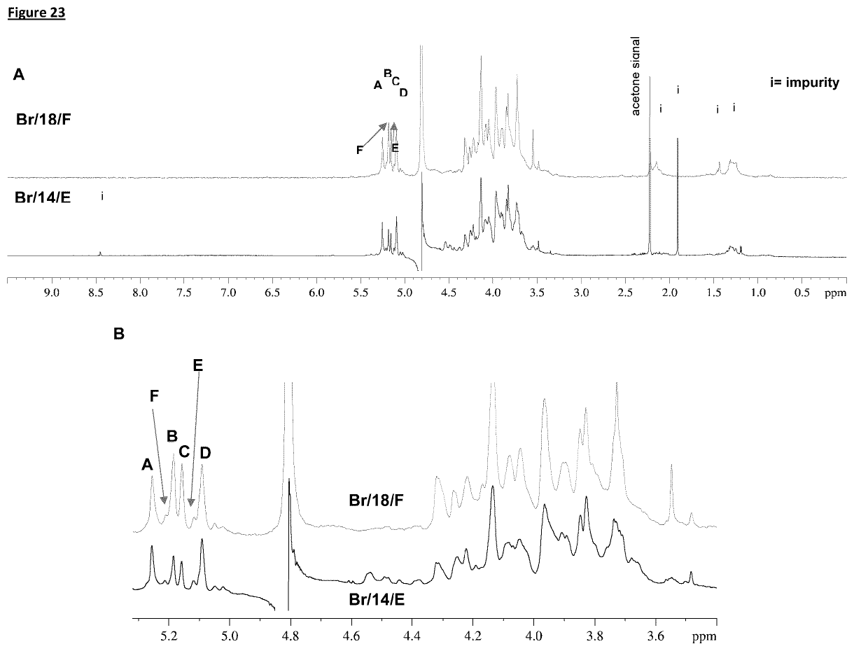

FIG. 23 shows (A) ¹H NMR spectra (600 MHz, 298 K, D₂O) of Br/14/E (less pure fraction) and Br/18/F (pure arabinan); and (B) Expansion of the ¹H NMR spectra of Br/14/E and Br/18/F. The structure of the arabinan is shown in FIG. 5.

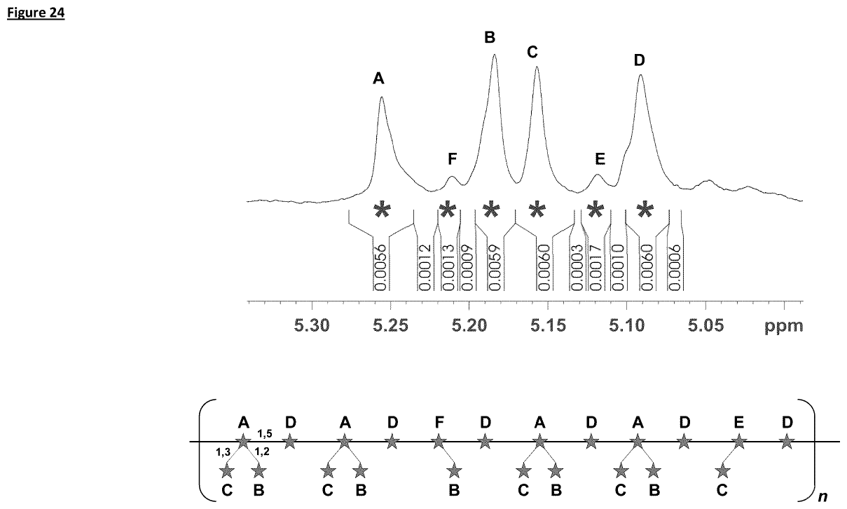

FIG. 24 (600 MHz, 298 K, D₂O) A) expansion of the ¹H NMR of the arabinan (Br/18/F) detailing the anomeric region along with the integration values used to evaluate the ratio between the different signals. Each signal is labelled with a capital letter and it is diagnostic of an arabinofuranose unit. B) structure of the arabinan polysaccharide drawn according to the SNFC rules, this structure includes the arabinofuranose units found by NMR analysis and maintains in good approximation the ratios detected by integration.

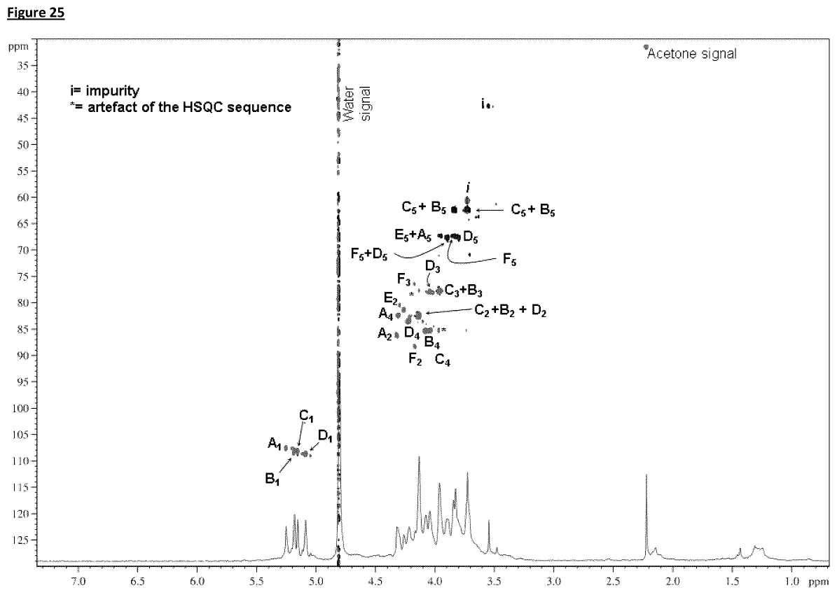

FIG. 25 (600 MHz, 298 K, D₂O) HSQC spectrum drawn at full scale of Br/18/F (pure arabinan with strong activity), densities are labelled for a quick identification, the proton spectrum is reported in red; for the structure of the arabinan and for the labels used refer to FIG. 5. “i” impurity, * artifact of the HSQC sequence.

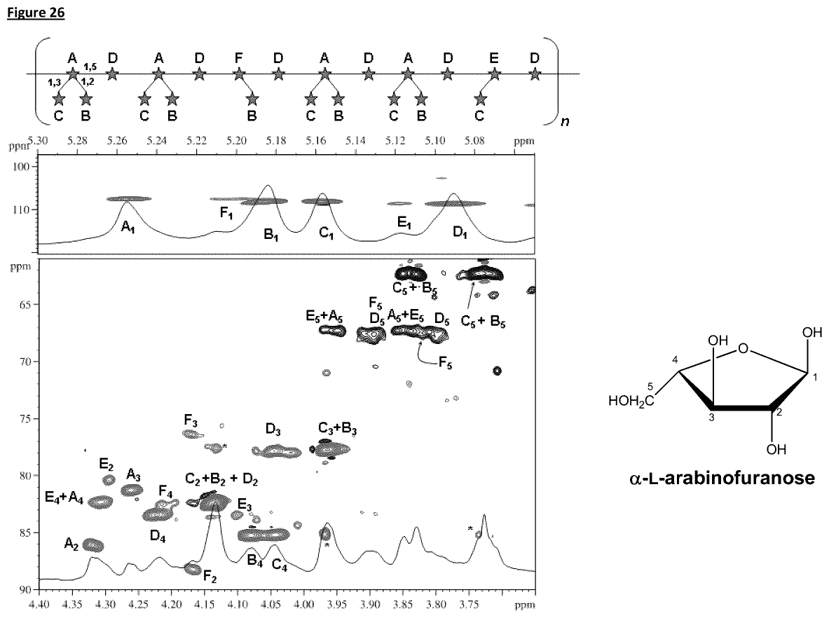

FIG. 26 (600 MHz, 298 K, D₂O) Expansion of the ¹H-¹³C HSQC spectrum of Br 18/F (pure arabinan with strong activity) detailing the (top) anomeric region, and (bottom) the carbinolic region along with the proton traces. The CH₂ groups (black) are distinguished from the other densities because the spectrum has been acquired in the DEPT mode. For the structure of the arabinan and for the labels used refer to FIG. 5.

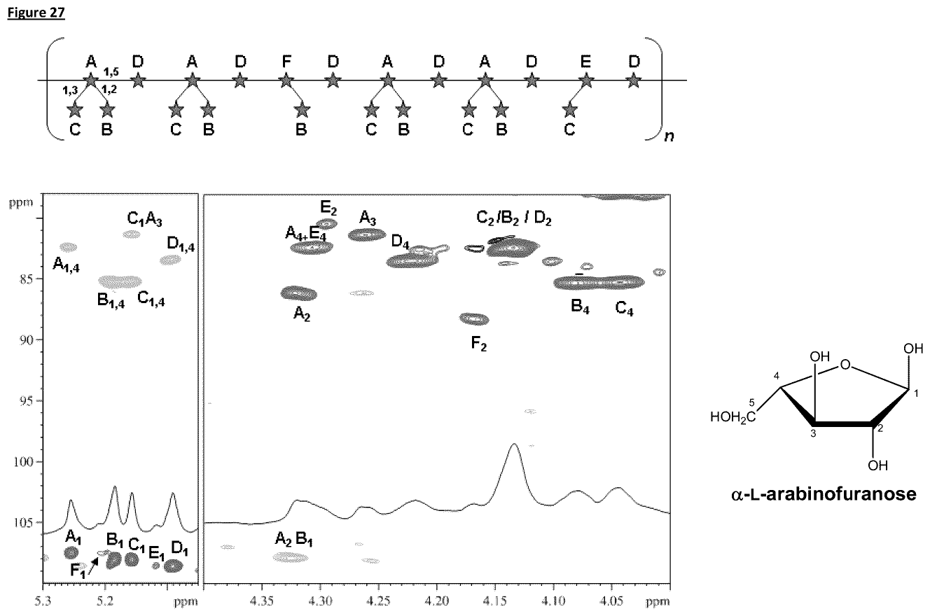

FIG. 27 (600 MHz, 298 K, D₂O) Selected regions of the NMR spectra reporting the overlap of the HSQC (dark grey) and HMBC (light grey) spectra of Br 18/F (pure arabinan with strong activity): anomeric (left panel) and carbinolic region (right panel). For the structure of the arabinan and for the labels used refer to FIG. 5.

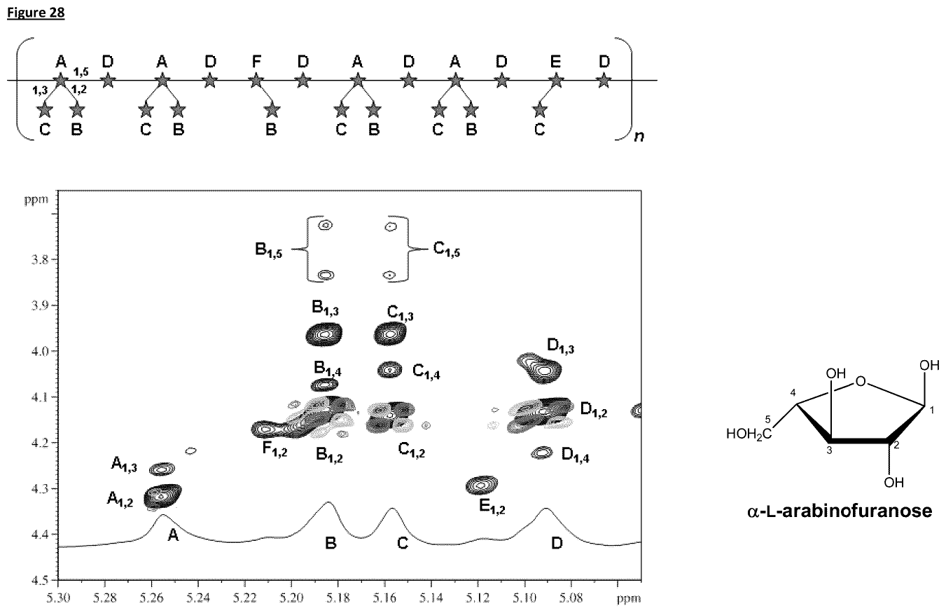

FIG. 28 (600 MHz, 298 K, D₂O) Expansion detailing the anomeric region of the arabinan (Br/18/F, the pure arabinan with strong activity) and reporting the overlap of the TOCSY (black) and COSY (dark and light grey) spectra. For the structure of the arabinan and for the labels used refer to FIG. 5.

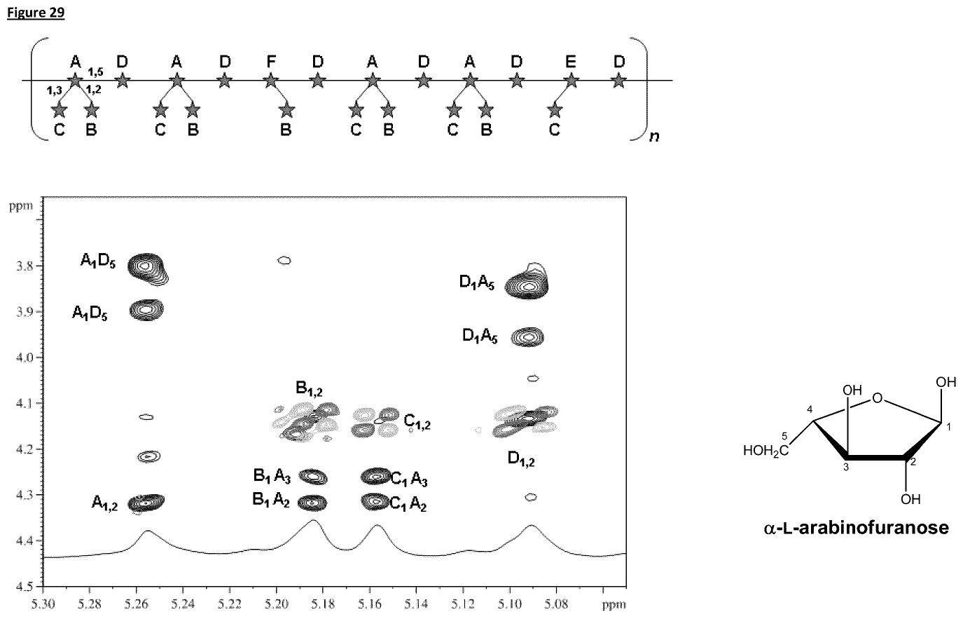

FIG. 29 (600 MHz, 298 K, D₂O) Expansion detailing the anomeric region of the arabinan (Br/18/F, the pure arabinan with strong activity) and reporting the overlap of the NOESY (black) and COSY (dark and light grey) spectra. For the structure of the arabinan and for the labels used refer to FIG. 5.

### EXAMPLES

### Materials and Methods

### Animals

Male APP_{swe}/PS1_{Δe9} (APP/PS1) mice with a C57B1/6J background were bred with wild-type C57B1/6J females at the Biomedical and Behavioural Research Unit at Ulster University in Coleraine. Offspring were ear punched and positivity for the APP_{swe}/PS1_{Δe9} transgene, or lack thereof was confirmed by polymerase chain reaction, using primers specific for the APP sequence of the APP/PS1 construct (Forward “GAATTCCGACATGACTCAGG” (SEQ ID NO: 1), Reverse: “GTTCTGCTGCATCTTGGACA” (SEQ ID NO: 2)). At 7 months of age, offspring males heterozygous for the APP_{swe}/PS1_{Δe9} transgenic construct were then age-matched and divided into two treatment groups. Mice in both groups were caged individually and allowed access to food and water ad libitum. Animals were maintained on a 12:12 light-dark cycle (lights on at 08 h00, lights off at 20 h00), within a temperature-controlled room (T: 21.5° C.±1° C.). All tests were performed during the light cycle. All experiments were licensed according to UK Home Office regulations (UK Animals Scientific Procedures Act 1986) and EU laws.

### Treatment

Seven month-old APP/PS1 mice were treated with saline (0.9% w/v) or a plant polysaccharide (PP) isolated from Sida cordifolia (300 mg/kg bw) once-daily by oral gavage for 3 weeks prior to commencement of a 12 day period of behavioural testing, during which time treatment was maintained. Mice in both groups were treated for a total of 33 days before sacrifice.

### Behavioural Analysis

Behavioural testing took place during the final 12 days of the study. Mice were subjected to open field, novel object recognition, Morris water maze and reversal water maze behavioural tests to assess locomotor function, anxiety and cognition in 7 month-old APP/PS1 mice treated with saline and PP.

### Immunohistochemistry

At the end of the study, subsequent to behavioural testing, mice were sacrificed and their brains were harvested and processed for immunohistochemical analysis, as described in. In both groups, brain sections were stained with GFAP, Iba1, 8-oxoguanine and IRS-1pSer⁶¹⁶ and levels of immunopositivity were quantified.

### Statistical Analysis

Data were analysed using Graphpad Prism (V6.0h) software (La Jolla, CA, USA). Statistical comparisons were made between 7 month-old APP/PS1 mice treated with PP and age-matched saline-treated APP/PS1 mice. Statistical tests used to generate the results presented in the following chapter include, ordinary one-way analysis of variance (ANOVA), ordinary and repeated measures two-way ANOVA and unpaired Student's t tests. Corrections for multiple comparisons were performed using Holm-Šídák's, Bonferroni's and Dunnett's post-hoc tests, where appropriate. P values smaller than 0.05 were considered statistically significant and data are expressed as means⊥SEM.

### Methods of Isolating the Plant Polysaccharide

The plant polysaccharide for use in the invention may be isolated from a Malvales plant, the method may comprise:
i. homogenising and dehydrating the Malvales plant, thereby forming dehydrated Malvales plant particles or powder; and
ii. extracting the polysaccharide from the dehydrated Malvales plant particles and/or powder, thereby isolating the polysaccharide from the Malvales plant.

The method may comprise isolating the polysaccharide from a member of the Malvales order or a part of the plant thereof (such as the roots). Thus, the Malvales plant or part thereof may comprise a plant of the Bixaceae family, the Cistaceae family, the Cytinaceae family, the Dipterocarpaceae family, the Muntingiaceae family, the Neuradaceae family, the Sarcloaenaceae family, the Sphaerosepalaceae family, the Thymelaeceae family or the Malvaceae family.

Preferably the plant is a member of the Malvaceae family. Thus, the member of the Malvales plant or part thereof may be a subfamily member of the family Malvaceae. The Malvaceae subfamilies comprise Bombacoideae, Brownlowioideae, Bytnnerioideae, Byttnerioideae, Dombeyoideae, Grewioideae, Helicteroideae, Malvoideae, Sterculioideae and Tiliodeae. Preferably the plant is a Malvoideae. The Malvoideae plant may be from the Malveae tribe, the Gossypieae tribe, the Hibisceae tribe or the Kydieae tribe. Preferably the plant is from the Malveae tribe. The plant may be from the Sida genus or the Malva genus or the Malvastrum genus or the Sidalcea genus or the Abufilon genus or the Althea genus or the Sphaeralcea genus or the Lavatera genus. Most preferably the plant of the Sida genus is Sida cordifolia (also referred to as ilima, flannel weed, bala, country mallow or heart-leaf sida). Most preferably the plant of the Malva genus is Malva sylvestris. Most preferably the plant of the Malvastrum genus is Malvastrum laterifium. Most preferably the plant of the Sidalcea genus is Sidalcea malviflora. Most preferably plant of the Abufilon genus is Abufilon theophrasfi. Most preferably the plant of Althea genus is Althea officinalis. Most preferably the plant of Sphaeralcea genus is Sphaeralcea coccinea. Most preferably the plant of Lavatera genus is Lavatera arborea. Most preferably the plant is Sida cordifolia.

The polysaccharide may be isolated from a plant of the Sida genus (e.g. Sida cordifolia) or the Malva genus (e.g. Malva sylvestris) or the Malvastrum genus (e.g. Malvastrum laterifium) or the Sidalcea genus (e.g. Sidalcea malviflora) or the Althea genus (e.g. Althea officinalis), or the Sphaeralcea genus (e.g. Sphaeralcea coccinea), or the Lavatera genus (e.g. Lavatera arborea). Preferably the polysaccharide is a Sida cordifolia polysaccharide. The polysaccharide may be isolated from part of Malvales plant, such as the leaves, the flowers, the stem and/or the roots of the plant. Preferably, the polysaccharide is isolated from the roots of the Malvales plant. The polysaccharide may be isolated from the roots of a Sida spp. (e.g. Sida cordifolia) or a Malva spp. (e.g. Malva sylvestris) or a Malvastrum spp. (e.g. Malvastrum laterifium) or a Sidalcea spp. (e.g. Sidalcea malviflora). Most preferably the polysaccharide is isolated from the roots of Sida cordifolia.

The step of homogenising and dehydrating the Malvales plant may comprise dehydrating the plant before homogenising the plant material, or dehydrating the plant after homogenising the plant. Homogenising the Malvales plant increases the surface area and/or reduces the size of the plant, such that the Malvales plant forms particles or a powder. Dehydrating removes moisture from the Malvales plant.

Homogenising the Malvales plant may comprise grinding and/or chopping the plant into particles and/or a powder. Homogenising the plant may comprise using an analytical mill. The particles or powder may be small enough to pass through a sieve size equal to or less than about 0.8 mm.

The step of dehydrating the Malvales plant may comprise lyophilising or heating the plant material to remove moisture. Preferably dehydrating comprises lyophilising.

The step of extracting the polysaccharide from the Malvales plant may comprise one or more from the group consisting of water-alcohol precipitation, dilute alkali leaching, enzyme treatment, microwave extraction, ultrasonic extraction, ultrasonic assisted enzyme extraction, vacuum extraction and pulsed electric field extraction. Preferably the step of extracting the polysaccharide comprises water-alcohol precipitation.

The step of extracting the polysaccharide from the dehydrated Malvales plant particles may comprise one or more alcohol extraction steps (e.g. ethanol extraction steps), which isolate the dehydrated Malvales plant particles or powder into an alcohol phase and a particulate phase, or an aqueous phase into an alcohol phase and a precipitate. The step of extracting the polysaccharide from the dehydrated Malvales plant particles may comprise one or more aqueous extraction steps, which isolate the dehydrated Malvales plant particles into aqueous phase and a precipitate, or which isolate an alcohol phase into an aqueous phase and a precipitate.

The step of extracting the polysaccharide from the dehydrated Malvales plant particles may comprise one or more alcohol extraction steps and/or one or more aqueous extraction steps.

The aqueous extraction step may be performed using an aqueous solution, such as water, saline or other solutions containing a salt (e.g. Phosphate Buffered Saline). The alcohol extraction step(s) may be performed using ethanol. Preferably there are two alcohol extraction steps, most preferably there is a first alcohol extraction step (e.g. an ethanol extraction step), followed by one or more aqueous extraction steps, followed by a second (final) alcohol extraction step (e.g. an ethanol extraction step).

The (first) alcohol extraction step may comprise isolating the dehydrated Malvales plant particles or powder into an alcohol phase and a particulate phase. Preferably the alcohol extraction step is performed for at least about 4, at least about 8 hours or at least about 12 hours, optionally while simultaneously stirring during the entire alcohol extraction step. The alcohol phase and the precipitate may be separated by centrifugation. Centrifugation may be performed at about 6000 rpm to about 7000 rpm, preferably about 6500rpm. Centrifugation may be performed at under about 0.1 to about 10° C., at about 1° C. to 9° C., at about 2° C. to 8° C., at about 2° C. to 7° C., at about 3° C. to 6° C. or at about 3° C. to 6° C. Preferably centrifugation is performed at about 4° C. Centrifugation may be performed for about 10 minutes or more, about 20 minutes or more, about 30 minutes or more, 1 hour or more. Preferably the centrifugation is performed for about 30 minutes or more. Centrifugation may be performed at about 6000 rpm to about 7000 rpm or at about 6500 rpm, at about 4° C., for about 10 minutes or more, for about 20 minutes or more, for 30 minutes or more. The alcohol phase may be discarded. The particulate phase, which contains the polysaccharide according to the invention, may be kept. The (first) alcohol extraction step may be performed using 95% alcohol (e.g. ethanol) at room temperature.

The one or more aqueous extraction steps may be one or more, two or more, three or more, four or more, five or more, six or more aqueous extraction steps. The one or more aqueous extraction steps may comprise isolating the precipitate of a first alcohol extraction step or an aqueous extraction step into an aqueous phase and a precipitate. The one or more aqueous extraction steps may be performed using a boiling aqueous solution, preferably an aqueous solution that has been maintained at a temperature of about 100° C. (during the entire extraction step). The aqueous extraction step may be performed for about 30 minutes or more, about 1 hour or more, or about 2 hours or more using a boiling aqueous solution, such as water. The aqueous extraction step may comprise stirring during the entire aqueous extraction step. The aqueous phase and the precipitate may be separated by centrifugation. Centrifugation may be performed at about 6000 rpm to about 7000 rpm, preferably about 6500rpm. Centrifugation may be performed at under about 0.1 to about 10° C., at about 1° C. to 9° C., at about 2° C. to 8° C., at about 2° C. to 7° C., at about 3° C. to 6° C. or at about 3° C. to 6° C. Preferably centrifugation is performed at about 4° C. Centrifugation may be performed for about 10 minutes or more, about 20 minutes or more, about 30 minutes or more, 1 hour or more. Preferably the centrifugation is performed for about 30 minutes or more. Centrifugation may be performed at about 6000 rpm to about 7000 rpm or at about 6500 rpm, at about 4° C., for about 10 minutes or more, for about 20 minutes or more, for 30 minutes or more. The separated aqueous phase of the one or more aqueous extraction steps, which contains the polysaccharide according to the invention, may be kept and pooled together. The precipitate of the aqueous extraction step may be used in a further aqueous extraction step to isolate further polysaccharide according to the invention.

The aqueous phase of the one or more aqueous extraction steps that have been pooled together may be treated with one or more enzymes at room temperature (or at about 37° C.) to digest unwanted carbohydrates and proteins, optionally followed by dialysis. The enzymes may be selected from the group consisting of alpha-amylase, pullulanase and protease K. Dialysis may be performed using water. The dialysis may have a pocket cut-off of about 12-14 kDa.

The second/final alcohol extraction step may be performed on the aqueous phase of the one or more aqueous extraction steps that have been pooled together, such that an alcohol phase and a precipitate are formed. Preferably the second/final alcohol extraction step is performed on the aqueous phase of the one or more aqueous extraction steps that have been pooled together, enzymatically treated and dialysed The (second/final) alcohol extraction step may be performed using 80% alcohol (e.g. 80% ethanol) at room temperature (e.g. about 18-22° C.). Preferably the alcohol extraction step is performed for at least about 4 hours, at least about 8 hours or at least about 12 hours, optionally while stirring during the entire alcohol extraction step. The alcohol phase and the precipitate may be separated by centrifugation. Centrifugation may be performed at about 6000 rpm to about 7000 rpm, preferably about 6500rpm. Centrifugation may be performed at under about 0.1 to about 10° C., at about 1° C. to 9° C., at about 2° C. to 8° C., at about 2° C. to 7° C., at about 3° C. to 6° C. or at about 3° C. to 6° C. Preferably centrifugation is performed at about 4° C. Centrifugation may be performed for about 10 minutes or more, about 20 minutes or more, about 30 minutes or more, 1 hour or more. Preferably the centrifugation is performed for about 30 minutes or more. Centrifugation may be performed at about 6000 rpm to about 7000 rpm or at about 6500 rpm, at about 4° C., for about 10 minutes or more, for about 20 minutes or more, for 30 minutes or more. The alcohol phase may be discarded. The particulate phase, which contains the polysaccharide according to the invention, may be used or further enriched using chromatographic techniques known in the art. Chromatographic techniques known in the art include ion-exchange chromatography, size-exclusion chromatography, reversed phase chromatography, high performance liquid chromatography and flash chromatography.

The method may comprise a final (iii) purification step. The purification step may comprise purifying the polysaccharide isolated from the Malvales plant using a chromatographic technique, a crystallisation technique or a distillation technique.

### Example—Isolation of the plant polysaccharide

The active arabinan polysaccharide of the invention can be isolated from plant material in a number of ways. An optimised 3-step extraction process (outlined in FIG. 20) leads to active arabinan residing in Br/29/16 (Br/29/17 is inactive). The active arabinan is present and highly enriched in Br/29/16 and is relatively pure but may be further purified by chromatographic methods.

Step I—initial extraction takes place with an overnight incubation of powered plant material in 95% ethanol at room temperature. Extraction is completed by centrifugation to obtain the precipitate pellet at the bottom of the tube. The remaining ethanol phase (Br/29/2) is discarded.

Step II—further extraction takes place by 6 repeated cycles of adding water to the pellet, boiling this (1 h;100° C.) using an oil bath and a thermocouple, then centrifugation (see details in FIG. 1). Each time the water phases (e.g. Br/29/3, 5, 7, 9, 11 and 14) are combined together, and the pellet (e.g. Br/29/4, 6, 8, 10 and 12) at the bottom of the tube undergoes a further extraction cycle.

Step III—the pooled extract is reduced in volume by a rotary evaporator. This undergoes enzymatic digestion (37° C.) to remove any unwanted polysaccharides and proteins (FIG. 1). The added enzymes include pullulanase (EC 3.2.1.41; Sigma Aldrich); α-Amylase (EC 3.2.1.1; Sigma Aldrich) and Protease k (EC 3.4.21.14; Sigma Aldrich). This digestate is then dialysed (Spectra/Por®4 Dialysis Membrane Standard RC Tubing; MWCO: 12-14 KDa; Thermo Fisher), and the final polysaccharide preparation (Br/29/16) is obtained by precipitation with ethanol and by a final centrifugation step. This pellet has potent immunomodulatory activity.

Determining the chemical structure of the plant polysaccharide in the active fraction of Sida cordofolia, and optimisation of the extraction/purification methodology.

An extract from the roots of Sida cordofolia was purified by gel-filtration, HPLC and other chromatography methods specified. A process of bioactivity-guided fractionation was undertaken to identify highly purified polysaccharides fractions with activity. Several different extraction/purifications strategies were undertaken and the resulting polysaccharides were evaluated in terms of: (i) activity, (ii) yield, (iii) purity, and (iv) molecular weight. Activity was determined by measuring NO responses in a macrophage cell line as previously described. Yield was determined by comparing dried weight of fractions against initial dry weight. Purity was assessed by NMR or chromatography as appropriated. The molecular weight was determined by SEC-HPLC (TSK gel G5000 PWXL, 30 cm×7.8 mm ID). The purified arabinan polysaccharides (30 μl; 1 mg/ml solution) were injected on the TSK column and eluted with 100% of 50 mM ammonium bicarbonate (Flow 0.8 ml/min). The eluate was monitored by refractive index. The column was calibrated with dextrans of known molecular weight (5 KDa, 50 KDa, 150 KDa, 410 KDa, 610 KDa) and the data fitted in a linear regression and the molecular weight evaluated accordingly (FIG. 21). The purified arabinan polysaccharide with strong immunomodulatory activity underwent:
quali-quantitative analysis of the monosaccharide components by CG-MS;
site of linkage analysis of the monosaccharide components (see FIGS. 22); and

1D and 2D NMR execution and analysis of the pure polymer (see FIGS. 23-29).

TABLE 2
Structure of the labelled residues.

| Label | Residue |
|---|---|
| A | α-L-Araf-(1 |
| | ↓ |
| | 2) |
| | [→5)-α-L-Araf-(1→5)-α-L-Araf- |
| | (1→]ₙ |
| | 3) |
| | ↑ |
| | α-L-Araf-(1 |
| B | α-L-Araf-(1 |
| | ↓ |
| | 2) |
| | [→5)-α-L-Araf-(1→5)-α-L-Araf- |
| | (1→]ₙ |
| | 3) |
| | ↑ |
| | α-L-Araf-(1 |
| C | α-L-Araf-(1 |
| | ↓ |
| | 2) |
| | [→5)-α-L-Araf-(1→5)-α-L-Araf- |
| | (1→]ₙ |
| | 3) |
| | ↑ |
| | α-L-Araf-(1 |
| D | α-L-Araf-(1 |
| | ↓ |
| | 2) |
| | [→5)-α-L-Araf-(1→5)-α-L-Araf- |
| | (1→]ₙ |
| | 3) |
| | ↑ |
| | α-L-Araf-(1 |
| E | [→5)-α-L-Araf-(1→5)-α-L-Araf- |
| | (1→]ₙ |
| | 3) |
| | ↑ |
| | α-L-Araf-(1 |
| F | α-L-Araf-(1 |
| | ↓ |
| | 2) |
| | [→5)-α-L-Araf-(1→5)-α-L-Araf- |
| | (1→]ₙ |

The linkage pattern of the arabinan (Br/14/E, less pure fraction) was determined according to De Castro et al (2010). Briefly, the sample (0.5 mg) was solved in DMSO (1 mL), treated with powdered NaOH, methylated with iodomethane (300 μL), hydrolyzed (2 M TFA, 200 μL, 120° C., 2 h), carbonyl-reduced with NaBD₄ (5 mg), and finally acetylated with acetic anhydride (50 μL) in pyridine (100 μL).

The PMAA derivatives were analyzed by GC-MS with an Agilent instrument (GC instrument Agilent 6850 coupled to MS Agilent 5973), equipped with a SPB-5 capillary column (Supelco, 30 m×0.25 i.d., flow rate, 0.8 mL min⁻¹) and He as carrier gas. Electron impact mass spectra were recorded with an ionization energy of 70 eV and an ionizing current of 0.2 mA. The temperature program used for all the analyses was the following: 150° C. for 5 min, 150→280° C. at 3° C./min, 300° C. for 5 min.

### NMR Acquisition Parameters

NMR analyses were performed on a Bruker 600 MHz equipped with a cryogenic probe and spectra were recorded at 298 K. Acetone was used as internal standard (¹H 2.225 ppm, ¹³C 31.45 ppm) and 2D spectra (¹H-¹H DQF-COSY, NOESY, ¹H-¹H NOESY, ¹H-¹H TOCSY, ¹H-¹³C HSQC and ¹H-¹³C HMBC) were acquired by using Bruker software (TopSpin 2.0). Homonuclear experiments were recorded using 512 FIDs of 2048 complex with 32 scans per FID, mixing time of 100 and 200 ms were used for TOCSY and NOESY spectra acquisition, respectively. HSQC and HMBC spectra were acquired with 512 FIDs of 2048 complex point, accumulating 90 scans each, respectively. Spectra were processed and analyzed using a Bruker TopSpin 3 program.

### Molecular Weight Determination of the Pure Arabinan Br/17/D

The molecular weight of the pure arabinan Br/17/D (equivalent of Br/18/F, see FIG. 21), was determined by SEC-HPLC (TSK gel G5000 PWXL, 30 cm×7.8 mm ID). In order to disclose the PM of the arabinan, 30 μl (1 mg/ml solution) were injected on the TSK column and eluted with 100% of 50 mM ammonium bicarbonate (Flow 0.8 ml/min). The eluate was monitored by refractive index. The column was calibrated with dextrans of known molecular weight (5 KDa, 50 KDa, 150 KDa, 410 KDa, 610 KDa), the data fitted in a linear regression and the molecular weight of Br/17/D evaluated accordingly (FIG. 21, Table 3).

TABLE 3
Molecular weight of the dextrans used to calculate the molecular
weight of the poly_1 and poly_2. Dextrans with molecular
weight from 5000 Da to 670000 Da were inject on the TSK column
(30 μl of a solution 1 mg/ml) and were used to construct
a calibration curve with the following straight-line equation:
y = −1.3958x + 15.176. Br/17/D was injected
on TSK column (30 μl of a solution 1 mg/ml) and the molecular
weight was determined by solving the straight-line equation.

| | MW(Da) | log MW | Time(min) | Elution volume |
|---|---|---|---|---|
| Dexstran | 5000 | 3.70 | 12.54 | 10.03 |
| Dextran | 50000 | 4.70 | 10.76 | 8.60 |
| Dexstran | 150000 | 5.18 | 9.90 | 7.92 |
| Dexstran | 410000 | 5.61 | 9.16 | 7.33 |
| Dexstran | 670000 | 5.8 | 8.85 | 7.08 |
| Br/17/D | 48977.88 | 4.69 | 10.79 | 8.63 |

### Reference

De Castro C, Parrilli M, Hoist O, Molinaro A. Microbe-Associated Molecular Patterns in Innate Immunity: Extraction and Chemical Analysis of Gram-Negative Bacterial Lipopolysaccharides. Methods Enzymol. 2010, 480, 89-115. Doi: 10.1016/S0076-6879(10)80005-9.

### Example A

Plant polysaccharide material may be obtained from roots of Tilia cordata, Sparrmannia africana, Dombeya wallichii, Lagunaria patersonii, Pachira aquatic, Hibiscus syriacus, Hibiscus waimeae, Hibiscus rosa sinensis, Pavonia spinifex, Abutilon theophrasti and Sidalcea malviflora were kindly collected and donated by Belfast Botanic Gardens. Specimens of Thebroma cacoa, and Lavatera arborea were kindly donated by the Royal Botanic Garden, Edinburgh. Sida cordifolia roots were donated by Pukka herbs, Bristol. Malva sylvestris, Sphaeralcea coccinea, Gossypium hirsutum, Gossypium herbaceum, Malvastrum lateritium were grown from seeds, and Althea officinalis roots were obtained from Neal Yard Remedies, London.

Fresh plant roots were washed with isopropanol and water, roots were subsequently lyophilised and stored at −20° C. Lyophilised roots were homogenised into a fine powder using an analytical mill and 100 g of each root was macerated successively in n-hexane, chloroform, methanol and ddH2O, at room temperature for 24 h, in a successive manner. The n-hexane, chloroform and methanol extracts were concentrated in vacuo, using a rotary evaporator set at 45° C., the aqueous extract was centrifuged at 2000 g for 15 minutes and subsequently lyophilised. A precipitate was isolated from lyophilised aqueous extracts by dissolving lg of aqueous extract in 10-20 ml ddH2O, followed by ethanol precipitation (abs.), the precipitate was pelleted by centrifugation which was later lyophilised and stored at −20° C., the isolated precipitate fractions were labelled at EXAP.

### Polysaccharide Analysis

The presence of polysaccharides in the alcohol precipitate was confirmed by the Molisch's reagent (5% thymol dissolved in alcohol (abs.). Briefly, 10 mg samples where dissolved in 750 μl of ddH2O in a glass test tube to which 500 μl of the Molisch's reagent was added, followed by the addition of 3 gtt of concentrated sulphuric acid.

### Determining Protein Content in Polysaccharide Enriched Fractions

As the method for precipitation of polysaccharide can also precipitate proteins. Therefore, the protein content in precipitate had to be determined. A number of methods have been developed for determining protein content in physiological fluids obtained from animal sources, unfortunately these assays have been developed based on protein-copper chelation, which results in the reduction of copper from Cu (II) to a Cu (I), and can be influenced by the presence of reducing agents that are present in plant extracts (Compton & Jones 1985). In contrast, the Bradford assay is based on the formation of a complex between Brilliant Blue G250 (Coomassie Brilliant Blue) dye and between basic amino acid residues (arginine, lysine and histidine). The resulting complex results in a shift in the absorption maximum of Brillant Blue G250 from λ 465 to 595 nm (Bradford 1976).

Briefly, polysaccharide enriched fractions were dissolved in PBS (0.5 mg/ml), 10 μl of the 0.5 mg/ml fractions were aliquoted in triplicate to wells of a 96-well plate in which 250 μl of the Bradford reagent (Bio-Rad) was added at room temperature. A protein standard of 1 mg/ml bovine serum albumin (BSA) (Sigma Aldrich) in PBS, which was subsequently serially diluted (1:10) to form standards ranging from 0-100 μg/ml. 10 μl was added to the wells of a 96-well plate in triplicate along with samples followed by 150 μlof the Bradford regent. The plate was incubated at room temperature for 20 min and absorbance measured at λ 595 nm, using a Tecan Safire 2 microplate reader (Tecan, Switzerland).

### Infra-Red Spectroscopy.

Raman-IR spectroscopy (Thermo Scientific Nicolet iS5 FT-IR Spectrometer with Omnic Software™, Madison, Wisconsin, USA) was performed followed by 13C NMR and 1H NMR in MeOH using a 400 MHz Bruker NMR (Billerica, MA, USA) to verify the structure.

### GC-MS Analysis of Polysaccharides

The polysaccharide enriched fractions (100 mg) were hydrolysed with 10 ml 1 M trifluroacetic acid (TFA) (Sigma-Aldrich) at 105° C. for 7 h in a closed 25 ml flask, (Uzaki & Ishiwatari 1983) and then subsequently lyophilised. Thereafter samples (2 mg) were derivitised by a silylation reaction with 500 μl N,O-Bis(trimethylsilyl)trifluoroacetamide (BSTFA) with 1% trimethylchrosilane (Sigam-Aldrich) in lml anhydrous pyridine. The reaction was carried out at room temperature for 12h.

GC-MS analysis was performed using gas chromatography (Agilent 7890A) interfaced 5 with a mass selective detector (Agilents 5975C), with a ZB semi-volatiles column (30m x 0.25mm x 0.25μm ZebronTM, Phenomenex Inc) with helium as the carrier gas at a constant rate of 1 ml/ min. The injector and MS source temperatures were maintained at 260° C. and 230° C., respectively. The column temperature program consisted of injection at 80° C. and hold for 1 min, temperature increase of 15° C. min-1 to 300° C., 10 followed by an isothermal hold at 300° C. for 15 min. The MS was operated in the electron impact mode with an ionisation energy of 70eV. The scan range was set from mass scan range was 50-550 Da. Injection volume was lμ1, inlet had a split flow of 20 ml-1 (split ratio 20:1).

15 Data was acquired and processed with the Chemstation software (Hewlett Packard).

Compound identification was performed by comparison with chromatographic retention characteristics and mass spectra of standards, and the NIST mass spectral library (National Institute of Standards and Technology, USA) (Magalhaes et al. 2007).

20

Example B Extraction of Plant Polysaccharide Material Sida cordifolia L radix was collected in Karnataka, India (2012), and donated by Pukka Herbs, Bristol, a voucher specimen was deposited in the DBN Economic Collections, Glasnevin 25 Herbarium Dublin (DBN 06:201261). Plant roots were washed with isopropanol and water. The lyophilised roots were homogenised to a fine powder using an IKA® All analytical mill (IKA® Werke GmbH & Co. KG, Staufen, Germany). Powdered (200 g) was macerated in n-hexane, chloroform, methanol and ddH20, at room temperature for 24 h, in a successive manner The n-hexane, chloroform and methanol extracts were concentrated in vacuo, using a rotary evaporator 30 set at 45° C., the aqueous extract was centrifuged at 4500 rpm for 15 minutes and subsequently lyophilised, extracts weighed 0.092g, 54.32 g 16.4 g, and 62.2g respectively. The polysaccharides in the lyophilised aqueous extract (62.2g) were extracted from the lyophilised aqueous extract by dissolving lOg in 20m1 ddH20, followed by ethanol precipitation (abs.) (1:4 v/v) overnight. The resulting precipitate was pelleted by centrifugation, and supernatant discarded. The yield of the 35 lyophilised pellet was 2.356 g (23.56% of lyophilised aqueous extract).

### Fractionation of Polysaccharides

The crude polysaccharide fraction obtained above (2.00 g) was further fractionated using DEAE™ Sephadex A-50 (weak anion exchanger) (GE Healthcare) column (30×2.5) pre-equilibrated with ddH20. Elution was stepwise using NaCl solutions of increasing ionic strength low (0 mol/l), medium (0.75 mol/l) and high (2 mol/l) NaOH. Resulting fractions were lyophilised and weighed, yielding 0.089 g, 0.251 g and 1.412 g, respectively. Fractions were further fractionated according to molecular size, using Vivaspin™ molecular weight cut-off (MWCO) filters (Sartorius, Goettingen Germany). Briefly, lyophilised extracts were dissolved at concentrations of 50 mg/ml in ddH20 and loaded onto a 100 kDa MWCO filter and centrifuged (2000 g; 1 h). Residue remaining in the 100 kDa MWCO filter was collected in a 1.5 ml Eppendorf tube by dissolving (200 μl) and rinsing (100 μl) with water. This procedure was repeated for all ion exchange chromatographic fractions (low, medium and high). Eluents from 10 kDa MWCO filters were collected but subsequently discarded once it was determined that all were inactive.

Fractions resulting from the above process were: SCAF0 (crude polysaccharide fraction of S. cordifolia); SCAF 1: low ionic strength and <100 kDa (56.1 mg); SCAF2: medium ionic strength and between 10-100 kDa (142.6 mg) SCAF 3 medium ionic strength and <100 kDa (72.5 mg); SCAF 4 high ionic strength (2 mol/l) and between 10 kDa-100 kDa (532.6 mg) and SCAF 5: high ionic strength and <100 kDa (786.7 mg).

### Results

Seven month old APP/PS1 mice were administered either saline (0.9% w/v) or a plant polysaccharide (PP) (300 mg/kg bw) by oral gavage, once daily (at 15:00 h) for 3 weeks prior to the commencement of a 12-day battery of behavioural tests, during which time, dosing was continued.

Locomotor Function in APP/PSI Mice Treated with Plant Polysaccharide

Assessment of spontaneous locomotor activity in the open field task showed that path length (FIG. 1A; t=0.03787, df=20, p=0.9702), speed (FIG. 1B; t=0.02828, df=20, p=0.9777) and linearity (FIG. 1C; t=0.3294, df=20, p=0.7453) were not significantly different in APP/PS1 mice that received PP, compared to those that were treated with saline, indicating a similar level of locomotor function between treatment groups.
Anxiety Levels in APP/PSI Mice Treated with Plant Polysaccharide

Similarly, defecation (FIG. 2C; t=0.6904, df=20, p=0.4979), rearing (FIG. 2A; t=0.1495, df=20, p=0.8826) and grooming (FIG. 2B; t=0.4201, df=20, p=6789) behaviour was not significantly different in treated with PP, compared to saline controls. Ordinary two-way ANOVA showed time spent in the centre of the arena was not significantly different in PP-treated APP/PS1 mice, compared to saline-treated controls (FIG. 2E; t=0.7122, df=86, p=0.4783). Together, this suggests that PP treatment had no effect on anxiety levels in APP/PS1 mice.

### Impaired Recognition Memory in APP/PSI Mice is Restored by PP Treatment

In the acquisition phase of the novel object recognition task, multiple t tests with Holm-Šídák's multiple comparisons test showed that the recognition indices for the identical objects were not significantly different in APP/PS1 mice treated with either saline (t=0.252671, df=22.0, p=0.8029) or PP (FIG. 3A; t=0.0558405, df=18.0, p=0.9561), indicating an absence of place preference in both treatment groups. In the test phase, multiple t tests with Holm-Šídák's post-hoc test demonstrated that saline-treated APP/PS1 mice spent a similar amount of time exploring the two objects and the recognition index for the novel object was not significantly different from the familiar (FIG. 3B; t=0.559631, df=22.0, p=0.5814). In APP/PS1 mice that received PP, the recognition index for the novel object was significantly higher than the familiar (FIG. 3B; t=4.20958, df=18.0, p=0.0005), indicated by ordinary multiple t tests with Holm-Šídák's multiple comparisons test. This suggests that although recognition memory is impaired in saline-treated control mice, treatment of APP/PS1 mice for 3 weeks with PP effectively restored this deficit.

### Spatial Learning and Memory are Improved by Plant Polysaccharide Treatment in APP/PSI Mice

The acquisition phase of the Morris water maze task took place over 4 consecutive days within the final 12 days of the study. Two-way repeated measures ANOVA showed that escape latency significantly decreased over time (F_{(3,258})=19.69, p<0.0001) and there was also a significant effect of treatment on escape latency, with an overall decrease detected in APP/PS1 mice treated with PP (F_{(1,86)}=0.1867, p=0.0122; FIG. 4A). Although, Bonferroni's multiple comparisons test indicated that average escape latency was not significantly different treatment groups, however there was a trend towards a decrease in escape latency on day 3 in PP-treated APP/PS1 mice, compared to saline-treated controls (FIG. 4A; p=0.0630). Two-way ANOVA found that path length decreased significantly over time (F_{(3,344)}=6.644, p=0.0002), but was not affected significantly by treatment (F_{(1,344})=0.8869, p=0.3470; FIG. 4B). Bonferroni's multiple comparisons post-hoc test confirmed that path length was not significantly different between saline- and PP-treated APP/PS1 mice on any of the Morris water maze trials days (FIG. 4B). Two-way repeated measures ANOVA showed that swim speed was significantly influenced by both time (F_{(3,344})=5.047, p=0.0020) and treatment (F_{(1,344})=5.491, p=0.0197), however Bonferroni's post-hoc test showed that swim speed did not differ significantly between treatment groups on any of the training days (FIG. 4C).

In the probe trial, PP-treated mice spent more time in the exact target area than saline controls, although this failed to reach significance (FIG. 4D; t=1.406, df=20, p=0.1751). Ordinary one-way ANOVA revealed that time spent in each of the probe quadrants by saline-treated APP/PS1 mice was significantly different (FIG. 4E; F_{(3,44)}=4.089, p=0.0121) and Dunnett's post-hoc test demonstrated that time in the target quadrant was significantly higher than the CCW quadrant (FIG. 4E; p<0.05). In mice that received PP, time spent in each of the probe quadrants was not significantly different (FIG. 4F; F_{(3,36)}=2.256, p=0.0985). These results suggest that PP treatment had minimal effects on spatial learning and memory in APP/PS1 mice in the Morris water maze task.

### Reversal Learning and Memory is Slightly Improved by Plant Polysaccharide Treatment in APP/PSI Mice

In the acquisition phase of the reversal water maze, two-way repeated measures ANOVA showed that there was a significant effect of time on escape latency (F_{(3,258)}=5.688, p=0.0009). Although significant differences in escape latency between treatment groups were not detected by Bonferroni's multiple comparisons test on any of the reversal training days, two-way repeated measures ANOVA showed that overall, escape latency in the reversal acquisition phase was significantly lower in PP-treated APP/PS1 mice (FIG. 5A; F_{(1,86)}=4.375, p=0.0394). A significant overall effect of time (F_{(3,258)}=6.189, p=0.0004) and treatment (F_{(1,86)}=6.469, p=0.0128) on path length was detected by two-way ANOVA in the reversal acquisition phase. Bonferroni's post-hoc test however showed that path length did not differ significantly in PP-treated, compared to saline-treated APP/PS1 mice (FIG. 5B). Two-way repeated measures ANOVA showed that neither time (F_{(3,258)}=0.4865, p=0.6920) nor treatment (F_{(1,86)}=1.689, p=0.1972) had a significant overall effect on swim speed in the reversal acquisition phase (FIG. 5C).

In the probe trial of the reversal water maze task, time in the exact target area was not significantly different between treatment groups (FIG. 5D; t=0.9123, df=20, p=0.3725). Ordinary one-way ANOVA showed that saline-treated APP/PS1 mice spent a significantly different amount of time in each of the reversal probe quadrants (F_{(3,44)}=3.359, p=0.0271) and Dunnett's multiple comparisons test showed that time in the target quadrant was significantly greater than the CW quadrant (FIG. 5E; p<0.05). Time spent by PP-treated mice in each of the reversal probe quadrants was also significantly different, as shown by one-way ANOVA (F_{(3,36)}=3.292, p=0.0314), however Dunnett's post-hoc test revealed that time in the target quadrant was not significantly different from the other quadrants (FIG. 5F).

### Sensorimotor Function in APP/PSI Mice is Unaffected by PP Treatment

On the final day of behavioural assessment, a marker was attached to the escape platform, which had been relocated to the quadrant opposite quadrant used for the reversal water maze and counterclockwise to the quadrant used in the Morris water maze and three visual trials were performed. Two-way repeated measures ANOVA indicated that visual escape latency (FIG. 6A; F_{(1,20)}=0.03816, p=0.8471), path length (FIG. 6B; F_{(1,60)}=0.006323, p=0.9369) and swim speed (FIG. 6C; F_{(1,60)}=0.01586, p=0.9002) were not significantly affected by treatment and Bonferroni's multiple comparisons tests detected no differences in escape latency, path length or speed between saline- and PP-treated groups in any of the 3 trials (FIGS. 6A-C). This suggests that sensorimotor function in APP/PS1 mice was unaffected by PP treatment.

### Plant Polysaccharide Treatment Reduces Aβ Deposition in the Brains of APP/PSI Mice

FIGS. 7A, B, D and E illustrate a reduction of AP deposition in the cerebral cortex and dentate gyrus of PP-treated APP/PS1 mice, compared to saline controls. Quantitative analysis revealed this reduction of AP immunopositivity to be significant in both the cortex (FIG. 7C; t=5.625, df=10, p=0.0002; 47% reduction) and dentate gyrus (FIG. 7F; t=2.995, df=10, p=0.0135; 65% reduction) of APP/PS1 mice treated with PP.

### Plant Polysaccharide Treatment Reduces Levels of Astrocytes in the Cerebral Cortex of APP/PSI Mice

A reduction in levels of GFAP-positive astrocytes was seen in the cortex of PP-treated APP/PS1 mice, as illustrated by representative micrographs (FIGS. 8A and B). Quantification revealed a significant 25% reduction of cortical GFAP immunopositivity in comparison to saline-treated APP/PS1 mice (FIG. 8C; t=2.649, df=10, p=0.0244). GFAP levels in the dentate gyrus, however were similar between PP- and saline-treated mice (FIGS. 8D and E) and quantification demonstrated that astrocyte levels in the dentate gyrus of PP-treated APP/PS1 mice were not significantly different from saline-treated control mice (FIG. 8F; t=0.2331, df=10, p=0.8204).

### Plant Polysaccharide Treatment Reduces Levels of Microglia in the Cerebral Cortex of APP/PSI Mice

Similarly, levels of Ibal-positive microglia were lower in the cerebral cortex of APP/PS1 mice treated with PP (FIGS. 9A and B) and, again quantitative analysis demonstrated that this reduction was significant in the cortex (FIG. 9C; t=2.604, df=10, p=0.0263; 16% reduction). Microglial levels were also similar in the dentate gyrus of PP-treated APP/PS1 mice, in comparison to saline controls (FIGS. 9D and E) and quantification was unable to detect a significant difference in Ibal immunopositivity between PP- and saline-treated APP/PS1 mice in the dentate gyrus (FIG. 9F; t=1.600, df=10, p=0.1408).

### Plant Polysaccharide Treatment Reduces Levels of Oxidative Stress in the Cerebral Cortex of APP/PSI Mice

Representative micrographs show that while a reduction of oxidative stress was seen in the cortex of APP/PS1 mice treated with PP (FIGS. 10A and B), 8-oxoguanine levels were similar between treatment groups in the dentate gyrus (FIGS. 10D and E). Quantification demonstrated that 8-oxoguanine levels were indeed significantly reduced by 24% in the cortex of PP-treated APP/PS1 mice, compared to saline controls (FIG. 10C; t=3.081, df=10, p=0.0116), while 8-oxoguanine immunopositivity was not significantly different in the dentate gyrus of PP-treated mice, compared to APP/PS1 mice that received saline (FIG. 10F; t=0.3781 df=10, p=0.7133).

Plant Polysaccharide Treatment has No Effect on Levels of IRS-1 pSer⁶¹⁶ in the Brains of APP/PSI Mice

Representative micrographs show that IRS-1pSer⁶¹⁶ levels were similar between treatment groups in both the cerebral cortex (FIGS. 11A and B) and dentate gyrus (FIGS. 11D and E). Quantification demonstrated that IRS-1pSer⁶¹⁶ immunopositivity was not significantly different in the cortex (FIG. 10C; t=1.117, df=10, p=0.2902) or dentate gyrus (FIG. 10F; t=1.431, df=10, p=0.1830) of PP-treated APP/PS1 mice, compared to those that received saline.

### Plant Polysaccharide Treatment has No Effect on Spleen Weight in APP/PSI Mice

Spleens were dissected from mice at the end of the study and weighed. Splenic weights in PP-treated APP/PS1 mice did not differ significantly from saline controls (FIG. 12; t=0.05859, df=20, p=0.9539), demonstrating that there was no spleen enlargement or inflammation as a result of treatment with PP in APP/PS1 mice.

### Example 5—The Effect of the Polysaccharide on Faecal Bacterial Diversity

Perturbations in the microbiota-gut-brain (MGB) axis have been linked to the development neuroinflammation, in the context of neurodegenerative disease, and decreased microbial diversity has been observed. As it is known that certain polysaccharides can increase beneficial gut microbiota, the effect of the isolated polysaccharide on microbial diversity was tested in wild-type and APP/PS1 mice.

Faecal matter of wild-type and APP/PS1 mice was assessed to determine bacterial diversity. Twelve-month-old APP/PS1 and wild-type mice were fed 300 μg of the polysaccharide, which had been isolated from Sida cordifolia, for 7 days. FIG. 13 shows the % composition of faecal microbiota in mice at the baseline and after 7 days of treatment with the polysaccharide. FIG. 14 is a summary of the data shown in FIG. 13 and also a summary of data in which bacterial diversity was analysed at the species level (Alpha diversity was calculated using the Shannon index). FIGS. 13 and 14 clearly show that the polysaccharide according to the invention increases bacterial faecal diversity at the phylum level and species level in both wild-type and APP/PS1 mice. Thus, the polysaccharide according to the invention can be used to treat neurodegenerative diseases and/or neuroinflammation by increasing microbial diversity in the gut.

### Example 6—The Polysaccharide Increases Plasma Levels of IL-27 in Wild-Type and APP/PS1 Mice and Reduces Plasma TNFα Levels in the Same Mice

Isolated plant polysaccharide (300 mg/kg) or saline vehicle control was orally administered to 8-month-old APP/PSI mice for 7 days. Cytokine levels of IL-27 were significantly increased in wild-type (p<0.05) and APP/PS1-polysaccharide-treated mice (p<0.05) compared to their respective controls. Furthermore, circulating IL-27 concentration in APP/PS1 polysaccharide-treated mice was significantly increased compared to wild-type saline animals (p<0.05). *p<0.05. TNF-α levels of were significantly reduced in wild-type PP-treated mice compared to saline-treated wild-types, and significantly reduced compared to saline (p<0.05), but not PP-treated APP/PSI animals. *p<0.05 ** p<0.001.

Given that decreased plasma concentrations of IL-27 in combination with increased plasma concentrations of TNFa correlate with increased disease severity in Parkinson's disease, and that the PP reverses the change in concentration of both cytokines (i.e., increases plasma IL-27 and decreases plasma TNF), the PP may be used to treat Parkinson's disease.

### Example 7—The Polysaccharide Potentiates Cytokines Responses to LPS Challenge

Neuroinflammation resulting from systemic infections has been shown to play an important role in neurodegenerative disease. LPS hypo-responsiveness has been associated with detrimental effects in brain, by inhibiting microglial protective properties thereby exacerbating neurodegenerative disease processes. Furthermore, systemic inflammation enhances the risk and progression of Alzheimer's disease, systemic infections are associated with a significant percentage of clinical relapses in multiple sclerosis, and systemic infections in PD enhance motor symptoms. The inventors therefore decided to investigate whether the polysaccharide according to the invention may has an effect on cytokine responses to intravenously administered LPS.

Isolated plant polysaccharide (PP) (300 mg/kg) or saline vehicle control was orally administered to 8-month-old APP/PS1 mice for 7 days. Cytokine levels of IL-10 (A), IL-27 (B), MCP-1 (C), IL-12p70 (D), IL-6 (E), GM-CSF (F), TNF-α(G) and I1-1β (H) and were measured on day 7, before administration of LPS (1 mg/kg; Pre-LPS), and 2.5 hours after administration of LPS (2.5 h post-LPS). In APP/PS1 polysaccharide-treated mice, IL-10, MCP-1, IL-12p7, IL-6, GM-CSF TNF-α and IL-1β responses were significantly enhanced compared to saline-treated APP/PS1 mice (P<0.05-P<0.01). In wild-type controls, PP administration potentiated MCP-1, IL-6 and TNF-a compared to saline-treated WT littermates (p<0.05-p<0.01). Increases in IL-10, IL-27, IL-12P70, GM-CSF and IL-1β were unique to APP/PS1 levels after administration of PP were unique to the APP/PSI model. *p<0.05, **p<0.01 and ***p<0.001.

In summary, this data shows that treatment with polysaccharide leads to much greater LPS responsiveness in the APP/PS1, and this represents a protective mechanism for the brain. Thus, the polysaccharide according to the invention can be used to potentiate neuroinflammatory responses and/or treat neurodegenerative disorders.

### Example 8—The Effect of the Polysaccharide on Cytokine Production by Microglia

The polysaccharide also causes microglia to produce NO in a concentration-dependent manner (see FIG. 18). Microglia produce IL-6 and IL-12 in a concentration-dependent manner in response to contact with the polysaccharide according to the invention but do not produce any detectable levels of TNFα (see FIG. 19).

1. An isolated polysaccharide (PP) comprising “n” repeating units; or
a composition comprising an isolated polysaccharide (PP) comprising “n” repeating units,
wherein each of the “n” repeating units comprises a backbone of alpha-(1-5)-linked arabinofuranose residues, a first side chain of a single alpha-arabinofuranose residue (1-2)-linked to an arabinofuranose residue of the backbone, and a second side chain of a single alpha-arabinofuranose residue (1-3)-linked to the same arabinofuranose residue of the backbone,
for use in treating, preventing or ameliorating a neurodegenerative disease and/or symptoms thereof in a subject,
optionally wherein the composition is a pharmaceutical composition and a pharmaceutically acceptable carrier or such like; or an edible composition.

2. A method of treating, preventing or ameliorating a neurodegenerative disease and/or symptoms thereof in a subject, the method comprising administering to the subject (i) an isolated polysaccharide comprising “n” repeating units; or
(ii) a composition comprising an isolated polysaccharide comprising “n” repeating units,
wherein each of the “n” repeating units comprises a backbone of alpha-(1-5)-linked arabinofuranose residues, a first side chain of a single alpha-arabinofuranose residue (1-2)-linked to an arabinofuranose residue of the backbone, and a second side chain of a single alpha-arabinofuranose residue (1-3)-linked to the same arabinofuranose residue of the backbone, or
(iii) a plant of the Malvales order or a part thereof

3. The polysaccharide for use according to claim 1 or the method according to claim 2, wherein the repeating units comprise or consist of Formula (I) (which may also be referred to herein as block “A”), defined herein as follows:

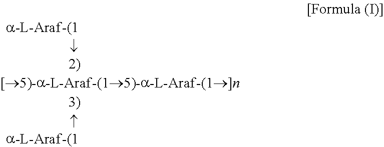

4. The polysaccharide for use according to any one of claim 1 or 3, or the method according to claim 2 or claim 3, wherein the repeating units further comprise block E, which is represented by Formula (II) as follows:

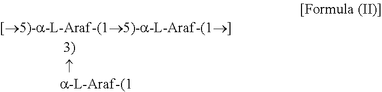

5. The polysaccharide for use according to any one of claim 1, 3 or 4 or the method according to any one of claims 2 to 4, wherein the repeating units further comprise block F, which is represented by Formula (III) as follows:

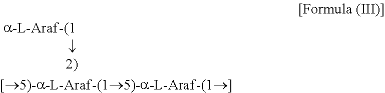

6. The polysaccharide for use according to any one of claims 3 to 5 or the method according to any one of claims 3 to 5, wherein the backbone of each block is linked by an alpha-(1-5)-glycosidic bond.

7. The polysaccharide for use according to any one of claims 3 to 5 or the method according to any one of claims 3 to 5, wherein the ratio of A:E:F in the repeating units is about 4-6:1-2:1.

8. The polysaccharide for use according to any one of claims 3 to 5 or the method according to any one of claims 3 to 5, wherein the ratio of A:F in the repeating units is about 4-6:1.

9. The polysaccharide for use according to any one of claims 3 to 5 or the method according to any one of claims 3 to 5, wherein the ratio of A:E in the repeating units is about 2-6:1.

10. The polysaccharide for use according claim 1 or the method according to claim 2, wherein the repeating units comprise Formula (IV), defined herein as follows: 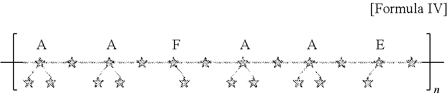
wherein each star of Formula (IV) corresponds to an arabinofuranose, preferably an L-arabinofuranose, more preferably an alpha-arabinofuranose, most preferably an alpha-L-arabinofuranose.

11. The polysaccharide for use according to any one of claims 1 and 3 to 10, or the method according to any one of claims 2 to 10, wherein “n” is about 5 to about 1000, about 10 to about 500, or about 15 to about 250, or about 15 to about 230, or about 15 to about 220. Preferably “n” is about 15 to about 220 or about 15 to about 230.

12. A plant of the Malvales order or a part thereof for use in treating, preventing or ameliorating a neurodegenerative disease and/or symptoms thereof in a subject.

13. A plant for use according to claim 12, wherein the plant of the Malvales order is Sida cordifolia.

14. A polysaccharide for use according to any one of claims 1 and 3 to 11 or the method according to any one of claims 2 to 11, the plant for use according to claim 12 or claim 13, wherein the neurodegenerative disease is one or more diseases selected from the group comprising or consisting of Alzheimer's disease (AD) and/or related dementias, such as vascular dementia, frontotemporal dementia and/or lewy body dementia; Parkinson's disease (PD) and/or PD-related disorders; Huntington's disease (HD); dementia; Motor neurone diseases (MND) (also referred to as amyotrophic lateral sclerosis (ALS)); Spinocerebellar ataxia (SCA); Spinal muscular atrophy (SMA); prion disease; autism spectrum disorders and/or the neurodegenerative process associated with autism spectrum disorders; depression; and schizophrenia.


SEQUENCE LISTING (2 sequences)

SEQ ID NO: 1 - DNA, 20 bp, synthetic construct
  source 1..20: mol_type = other DNA
gaattccgacatgactcagg

SEQ ID NO: 2 - DNA, 20 bp, synthetic construct
  source 1..20: mol_type = other DNA
gttctgctgcatcttggaca